(19) United States
(12) Patent Application Publication
(10) Pub. No.: US 2024/0108560 A1
(43) Pub. Date: Apr. 4, 2024

(54) HAIR DEMINERALIZER COMPOSITION, METHODS AND USES THEREOF

(71) Applicant: K18, Inc., San Francisco, CA (US)

(72) Inventors: Karis STALEY, Huntington Beach, CA (US); Meagan SCHLAPP, Maspeth, NY (US)

(21) Appl. No.: 18/478,320

(22) Filed: Sep. 29, 2023

Related U.S. Application Data
(60) Provisional application No. 63/378,021, filed on Sep. 30, 2022.

(51) Int. Cl. A61K 8/37; A61K 8/41; A61K 8/55; A61Q 5/00

(52) U.S. Cl. CPC A61K 8/375; A61K 8/41; A61K 8/55; A61Q 5/002; A61K 2800/522

(57) ABSTRACT

The present disclosure provides a composition and a method thereof for preventing and removing metal buildup accumulated in the hair. The hair demineralizer composition described herein comprises one or more chelating agents that are effective at binding metals in the hair under mild alkaline conditions, such as a pH from about 6 to about 9. The hair demineralizer composition may further comprise one or more antioxidants and/or one or more anti-redeposition agents that further assist in mitigating the effects of metal buildup accumulated in the hair.

### CROSS REFERENCE TO OTHER APPLICATION

This application claims priority to U.S. provisional application 63/378,021, filed Sep. 30, 2022, the entirety of which is incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Water hardness comes from dissolved salts of various metals including calcium (Ca²⁺) and magnesium (Mg²⁺). Hard water may also include trace metals. When hard water evaporates, the solid calcium carbonate CaCO₃, also known as scale, is formed. Water hardness levels vary significantly across the country and internationally as well.

### SUMMARY

In one aspect, the present disclosure provides compositions and methods for demineralizing hair. In some embodiments, the composition is a hair demineralizer composition comprising one or more demineralizing agents, and one or more antioxidants. In some embodiments, the composition comprises a pH from about 6 to about 9.

Hard water has adverse effects on the hair and directly impacts hair texture, At high concentrations, the various metal salts in hard water are difficult to wash out completely, causing mineral buildup in the hair. The various metals in hard water may react with chemicals in hair cleansing/treatment products, making the products less effective. In some instances, the metals interfere with uniformity of a bleach/lightener or color service, leading to undesirable and. streaky results. The mineral deposit buildup produced by the metals in hard water may combine with hair cleansing/treatment products to form contaminants that stick to the hair. Frequent washing with hard water may cause these minerals to build up, causing hair to have a rough and dry texture. The effects of the mineral deposit buildup can be exacerbated during chemical processing with peroxides in lightener, color, or perm services. Additionally, this mineral build up may cause the hair to look dull, have no shine, or appear hazy, Thus, there is an unmet need for a safe and effective demineralizer composition and methods for demineralizing the hair. Hair demineralizer compositions may restore hair conditions or improve hair treatments by removing minerals in hair.

In certain aspects, provided herein is a hair demineralizer composition, comprising: one or more demineralizing agents; and one or more antioxidants. In some embodiments, the hair demineralizer composition has a pH from about 6 to about 9.

In some embodiments, each of the one or more demineralizing agents binds to a primary metal, a secondary metal, or a combination of two or more thereof. In some embodiments, each of the one or more demineralizing agents binds a metal comprising calcium (Ca), magnesium (Mg), copper (Cu), iron (Fe), chromium (Cr), nickel (Ni), aluminum (Al), lead (Pb), zinc (Zn), cadmium (Cd), mercury (Hg), arsenic (As), barium (Ba), cobalt (Co), manganese (Mn), tin (Sn), tungsten (W), antimony (Sb), beryllium (Be), boron (B), bismuth (Bi), cesium (Ce), lithium (Li), molybdenum (Mo), selenium (Se), strontium (Sr), thallium (Tl), titanium (Ti), vanadium (V), scandium (Sc), gallium (Ga), yttrium (Y), niobium (Nb), technetium (Tc), ruthenium (Ru), rhodium (Rh), palladium (Pd), indium (In), tellurium (Te), rhenium (Rh), osmium (Os), or iridium (h), or a combination of two or more thereof. In some embodiments, the one or more demineralizing agents binds a metal selected from the group consisting of: calcium, magnesium, copper, iron, chromium, nickel, aluminum, lead, zinc, cadmium, mercury, arsenic, or a combination or two or more thereof.

In some embodiments, the hair demineralizer composition comprises at least 3 demineralizing agents. In some embodiments, the hair demineralizer composition comprises at least 4 demineralizing agents. In some embodiments, the hair demineralizer composition comprises at least 5 demineralizing agents.

In some embodiments, the one or more demineralizing agents is a chelator or sequestrant.

In some embodiments, the one or more demineralizing agents comprises trisodium dicarboxymethyl alaninate (MGDA), sodium phytate, phytic acid, sodium gluconate, tetrasodium glutamate diacetate (GLDA), tetrasodium ethylenediaminetetraacetic acid (EDTA), trisodium EDTA, disodium EDTA, pentasodiurn pentetate, trisodium ethylenediamine succinate, sodium thiosulfate, caprylhydroxamic acid, diiospropyl oxalate, disodium EDTA-copper, Hydroxyethylethylenediaminetriacetic acid (HEDTA), oxalic acid, potassium citrate, sodium citrate, sodium oxalate, tris(2-hydroxyethypammonium trihydrogen ethyl enediaminetetraacetate (TEA-EDTA), trisodium HEDTA, or a combination of two or more thereof.

In some embodiments, at least one of the one or more demineralizing agents comprises tetrasodium EDTA.

In some embodiments, the hair demineralizer composition comprises at least two demineralizing agents.

In some embodiments, the one or more demineralizing agents comprises trisodium MGDA, sodium phytate, sodium gluconate, tetrasodium GLDA, tetrasodium EDTA, or a combination of two or more thereof.

In some embodiments, the one or more antioxidants comprises carnosine, ascorbic acid, tetrahexyldecyl ascorbate, bioflavonoids, lycopene, Daucus carota saliva root cell culture lysate, monoammonium glycyrrhizinate, alpha lipoic acid (thioctic acid), Leontopodium alpinum (edelweiss) extract, Rosmarinus oificialis (rosemary) leaf extract, Vitis vinifera (grape) seed extract, Camellia sinensis leaf extract. Quercus robur wood extract, hydrolyzed olive fruit, Olea europea (olive) leaf extract, oleuropin, Punica granatum bark/fruit extract, punicalagin, ellagic acid, polyphenols, epigallocatechin (EGCG), tannins, sulforaphane, resveratrol, nordihydroguaiaretic acid, thioctic acid, or a combination of two or more thereof. In some embodiments, the one or more antioxidants is a peptide-derived antioxidant. In some embodiments, the peptide-derived antioxidant is carnosine, valine-cysteine, di-proline, di-phenylalanine, or a combination of two or more thereof.

In some embodiments, the hair demineralizer composition comprises trisodium MGDA, sodium gluconate, tetrasodium EDTA, and carnosine.

In some embodiments, the hair demineralizer composition has a pH of about 7 to about 8.

In some embodiments, the hair demineralizer composition comprises one or more surfactants. In some embodiments, the surfactant is a solubilizer. In some embodiments, the solubilizer is polysorbate 20, polyglyceryl-10 laurate, polyglyceryl-6 caprylate, polyglyceiyl-3 cocoate, polyglyceryl-4 caprate, polyglyceryl-6 ricinoleate, polyglyceryl-6 caprylate, sodium surfactin, or a combination of two or more thereof.

In some embodiments, the surfactant is a cleanser.

In some embodiments, the cleanser is Sapindus mukorossi peel extract, potassium cocoyl hydrolyzed oat protein, Starmella bombicola/brassica oil ferment, sophorolipids, canola sophorolipids, rhamnolipids, glycolipids, lauramidopropyl hydroxysultaine, lauryl hydroxysultaine, sodium lauroyl sarcosinate, sodium lauroyl isethionate, sodium cocoyl isethionate, lauroyl sarcosine, sodium lauroyl glutamate, sodium methyl cocoyl taurate, disodium cocoamphodiacetate, sodium C14-16 olefin sulfonate, sodium C14-16 alpha olefin sulfonate, benzethonium chloride, cocamidopropylamine oxide, decylamine oxide, decyl glucoside, lauryl glucoside, coca-glucoside, cocamidopropyl betaine, betaine, lauryl betaine, lauramidopropyl hydroxysultaine, cocamidopropyl hydroxysultaine, maltooligosyl glucoside, hydrogenated starch hydrolysate, or a combination of two or more thereof.

In some embodiments, the hair demineralizer composition comprises one or more pH modifiers. In some embodiments, the one or more pH modifiers is lactic acid, citric acid, malic acid, gluconic acid, glucuronic acid, glycolic acid, tartaric acid, azelaic acid, acetic acid, sodium hydroxide, triethanolamine, L-arginine, or a combination of two or more thereof.

In some embodiments, the hair demineralizer composition comprises one or more solvents.

In some embodiments, the one or more solvents comprises water, alcohols, dimethyl isosorbide, chlorotrifluoropropene, glycols, triethyl citrate, or a combination of two or more thereof.

In some embodiments, the hair demineralizer composition comprises one or more anti-redeposition agents. In some embodiments, the one or more anti-redeposition agents comprises an anionic functionalized biopolymer possessing sequestering capacity. In some embodiments, the one or more anti-redeposition agents is sodium carboxymethyl inulin, xanthan gum, hydroxyethylcellulose, hydroxymethylcellulose, cellulose, cellulose gum, microcrystalline cellulose, carboxymethylcellulose, pullulan, sclerotium gum, tetragonoloba (guar) gum, acacia senegal gum, hydroxypropyl starch phosphate, lithium magnesium sodium silicate, sodium magnesium fluorosilicate, or combinations thereof.

In some embodiments, the hair demineralizer composition further comprises one or more cosmetically or dermatologically acceptable excipients. In some embodiments, the one or more dermatologically acceptable excipients is a perfume, a preservative, or combinations thereof.

In some embodiments, the total concentration (%w/w) of the one or more demineralizing agents in the hair demineralizer is about 0.1% to about 15%.

In some embodiments, the total concentration (w/w%) of the one or more demineralizing agents in the hair demineralization is about 0.1% to about 5%.

In some embodiments, the total concentration (%w/w) of the one or more antioxidants in the hair demineralizer composition is about 0.1% to about 3%.

In another aspect, the present disclosure provides a method for demineralizing hair using the hair demineralizer composition disclosed herein.

In certain aspects, provided herein is a method for removing metals from hair comprising administering the hair composition as described herein.

Additional aspects and advantages of the present disclosure may become readily apparent to those skilled in this art from the following detailed description, wherein only illustrative embodiments of the present disclosure are shown and described. As may be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings which:

FIGS. 1A-E are bar graphs showing the average values for each element recorded on the four hair samples evaluated by TOF-SIMS (Example 1), according to sonic embodiments of the present disclosure.

FIGS. 2A-E show the average values for each element recorded on the four hair samples evaluated by TOF-SIMS (Example 2), according to some embodiments of the present disclosure.

FIGS. 3A-D show the average values for each element recorded on the four hair samples evaluated by TOF-SIMS (Example 3), according to some embodiments of the present disclosure.

FIG. 4 shows CIELAB system for measuring color, according to some embodiments of the present disclosure.

FIG. 5 shows delta a after 5 pool treatment cycles and the product use, according to some embodiments of the present disclosure.

FIG. 6 shows color change of cells 1 and 2 after soaking in contaminated hard water soak and bleach treatment, according to some embodiments of the present disclosure.

FIG. 7 shows color change of cells 3 and 4 after soaking in contaminated hard water soak and color treatment, according to some embodiments of the present disclosure.

### DETAWED DESCRIPTION

Water hardness comes from dissolved salts of various metals including calcium (Ca²⁺) and magnesium (Mg²⁺). Exemplary forms of these salts include bicarbonates (e.g., Ca(HCO₃)₂ or Mg(HCO₃)₂), sulfates (e.g., CaSO₄ or MgSO₄), and chlorides (e.g., CaCl₂ or MgCl₂). When hard water evaporates, solid calcium carbonate (CaCO₃), also known as scale, is formed. Water hardness levels are usually reported in grains per gallon (gpg), parts per million (ppm), or milligrams per liter (mg/L). Table 1 shows the water hardness levels and their representative concentrations.

TABLE 1
Water Hardness Levels

| Hardness level | Amount of metals (mg/L) | Amount of metals (gpg) |
|---|---|---|
| Soft | 0-17 | <1.0 |
| Slightly hard | 17-60 | 1.0-3.5 |
| Moderately hard | 61-120 | 3.5-7.0 |
| Hard | 121-180 | 7.0-10.5 |
| Very hard | >180 | >10.5 |

Water hardness levels vary significantly across the country and internationally. For example, Portugal has naturally soft water, while the UK has super hard water. Hardness exceeds 450 ppm (26 gpg) in certain regions of the Middle East.

In tap water (both hard and soft), trace metals are also present at lower levels. In some cases, the amount of trace metals is 10 to 100 times less than the amounts of Mg²⁺ and Ca²⁺. A nonlimiting example of trace metals includes iron (Fe), copper (Cu), zinc (Zn), aluminum (Al), lead (Pb), nickel (Ni), cadmium (Cd), chromium (Cr), mercury (Hg), arsenic (As), barium (Ba), cobalt (Co), manganese (Mn), tin (Sn), tungsten (W), antimony (Sb), beryllium (Be), boron (B), bismuth (Bi), cesium (Cs), lithium (Li), molybdenum (Mo), selenium (Se), strontium (Sr), thallium (Tl), titanium (Ti), vanadium (V), scandium (Sc), gallium (Ga), yttrium (Y), niobium (Nb), technetium (Tc), ruthenium (Ru), rhodium (Rh), palladium (Pd), indium (Iii), tellurium (Te), rhenium (Re), osmium (Os), and iridium (Ir). The elements on the edges of the Periodic Table, particularly metals of groups IA and IIA, prefer specific charges (e.g., ionized Ca and Mg tend to carry a positive charge of 2+), while those in the middle, of groups 1B-VIIB also known as the transition metals, can hold different charges (e.g., Fe²⁺, Fe³⁺, Fe⁴⁺, and Fe⁶⁺). As a result, these transition metals can be involved in various redox (oxidative-reductive) reactions.

Trace metals are found from sources other than water. Some of them play important biological roles as macro minerals. These metals include Ca, Mg, Fe, Cu, and Zn. Metals that are used or absorbed by the body may be removed in the hair formation process, where the metals end up inside hair fibers. Metal content in hair is an indicator of biological levels and, in some instances, hair is used in forensics to monitor environmental exposure to heavy metals. In some cases, metals are found in consumer products. Exemplary types of these metals include Cd, Ni, Pd, Fe, Cu, and Al. In some cases, metals are found in pollution. Exemplary of these metals include Hg, Pb, Cd, and Al from cigarette smoke.

Hair proteins naturally carry a net negative charge. In some cases, alkaline conditions and oxidative damage increase negative charges on hair, resulting in the potential for increased metal buildup because positively charged metal ions in water are attracted to and bind with the negatively charged hair proteins. These metals include Ca²⁺, Mg²⁺, and trace metal ions from hard and soft water. In some instances, sulfilr-containing amino acids (e.g., cysteine, methionine) act as binding sites for metal ions. When disulfide bonds are cleaved by chemical treatments, for example, perm treatment, free thiols are formed which bind the metal ions. In some instances, cysteic acid is formed by damaging oxidative chemical services forming additional binding sites for metal ions, Alkaline conditions may be encountered easily, as the average pH for household tap water in the US ranges from 6.5-9.5 and the majority of chemical services are performed at pH greater than 10, in cases some exceeding 12.

Depending on water source and quality, several factors may influence the binding of metals to hair. In some embodiments, the factors comprise pH, size of metal ion, concentration of metal ion, other metal ions present, state of metal ions (e.g., complexed salt or free form), and the condition of the hair. A general trend of binding affinity of metals to hair is Zn²⁺>Fe²⁺>Hg²⁺>Cu²⁺>Pb²⁺>Cr³⁺>Cd²⁺>Ni²⁺>Al²⁺. Additionally, hair has an affinity for heavy metals, which can damage human hair, and using hair as an alternative material for heavy metal decontamination of aqueous media has been proposed and studied.

The metal ions in hard water directly affect the texture of hair. For example, the metal ions create mineral buildup that interferes with hair cleansing and/or treatment and general manageability. As a result, the hair looks dull and has no shine, appears hazy and has a rough texture, or is dry and brittle. In some instances, metals interfere with uniformity of a bleach/lightener or color service, leading to undesirable and streaky results. In some instances, metal ions change the way various products sit on or interact with hair. In some cases, trace metal ions cause more significant damage. In some cases, trace metal ions catalyze oxidative reactions, further amplifying the incidental damage to hair by oxidative chemicals. In some cases, the effects of trace metal ions are exacerbated by sunlight (UV), atmospheric oxygen, pollution, or during chemical processing with peroxides in lightener, color, or perm services. In some instances, during the hair chemical treatment process, trace metal ions cause uneven and unpredictable color, smoking foils, or hair break-off. In some cases, metals alter or fade the color of hair. For example, a swimmer's green hair is a two-fold problem. Contrary to popular belief, the problem is not due to chlorine itself but to the presence of Cu²⁺ ions in the hair which impart the blue-green color, Chlorine, the active chemical in pool disinfectants, acts as a strong oxidizer which oxidizes the proteins in hair, increasing their ability to bind. Cu²⁺ ions. Further exposure to UV light, such as for example swimming outdoors, increases the damage and subsequently the number of negative charges in the hair, causing more Cu²⁺ to bind to the hair intensifying the greenish color of the hair.

Accordingly, there is a need for a demineralizer composition that can effectively and safely remove metal ion build up in the hair. More specifically, there is a need for a demineralizer composition which can effectively bind the various metal ions found in hard water including. calcium (Ca), magnesium (Mg), copper (Cu), iron (Fe), chromium (Cr), nickel (Ni), aluminum (Al), lead (Pb), zinc (Zn), cadmium (Cd), mercury (Hg), arsenic (As), barium (Ba), cobalt (Co), manganese (Mn), tin (Sn), tungsten (W), antimony (Sb), beryllium (Be), boron (B), bismuth (Bi), cesium (Cs), lithium (Li), molybdenum (Mo), selenium (Se), strontium (Sr), thallium (Tl), titanium (Ti), vanadium (V), scandium (Sc), gallium (Ga), yttrium (Y), niobium (Nb), technetium (Tc), ruthenium (Ru), rhodium (Rh), palladium (Pd), indium (In), tellurium (Te), rhenium (Re), osmium (Os), or iridium (Ir), or combinations of two or more thereof.

### Definitions

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art

Throughout this application, various embodiments may be presented in a range of format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example. 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises.” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “mixture” or “mixtures” is meant to include a simple combination of materials and any compounds that may result from their combination.

As used herein, the term “cosmetically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

As used herein, the term “cosmetically or dermatologically acceptable” refers to the compositions or components described that are suitable for use in contact with human skin tissue without undue toxicity, incompatibility, instability, allergic response, and the like.

As used herein, the terms “metal” and “metal ion” are used interchangeably to refer to an element of the Periodic Table of elements. Reference to a metal includes the different valences or oxidation state(s) of the metal. In some embodiments, for example, iron may refer to Fe, and may also refer to Fe⁺, Fe²⁺, Fe³⁺, Fe⁴⁺, or a mixture of Fe in its various oxidation states. For example, reference to Fe may include a mixture of Fe²⁺ and Fe⁴⁺, a mixture of Fe²⁺ and Fe³⁺, or a mixture of Fe²⁺, Fe³⁺, Fe⁴⁺, and so forth.

The section headings used herein are for organizational purposes only and are not to be construed as limiting the subject matter described.

### Hair Demineralizer Composition

Provided herein is a hair demineralizer composition for safely removing the various metal ions found in water from the hair. In some embodiments, the hair demineralizer composition comprises one or more demineralizing agents and one or more antioxidants. In some embodiments, the hair demineralizer composition has a pH from about 6 to about 9, or about 7 to about 8. In some embodiments, the one or more demineralizing agents bind to metal ions in the hair or water. In some embodiments, the one or more demineralizing agents binds to a metal ion in the hair that has been exposed to hard water. In sonic embodiments, the one or more demineralizing agents and the one or more antioxidants, work together to address the negative effects associated with metal build up in the hair.

In some embodiments, the hair demineralizer composition comprises one or more demineralizing agents, one or more antioxidants, and one or more pH modifiers. In some embodiments, the hair demineralizer composition comprises one or more demineralizing agents, one or more antioxidants, one or more pH modifiers, and one or more solvents. In some embodiments, the hair demineralizer composition comprises one or more demineralizing agents, one or more antioxidants, one or more pH modifiers, one or more solvents, and one or more surfactants. In some embodiments, the hair demineralizer composition comprises one or more demineralizing agents, one or more antioxidants, one or more pH modifiers, one or more solvents, one or more surfactants, and one or more anti-redeposition agents. In some embodiments, the hair demineralizer composition comprises one or more demineralizing agents, one or more antioxidants, one or more pH modifiers, one or more solvents, one or more surfactants, one or more anti-redeposition agents, and one or more cosmetically or dermatologically acceptable excipients. In some embodiments, the hair demineralizer composition comprises any combination of one or more demineralizing agents, one or more antioxidants, one or more pH modifiers, one or more solvents, one or more surfactants, one or more anti-redeposition agents, and one or more cosmetically and dermatologically acceptable excipients.

In some embodiments, the hair demineralizer composition comprises one or more demineralizing agents and one or more antioxidants. In some embodiments, the hair demineralizer composition comprises a pH of about 6 to about 9. In some embodiments, the hair demineralizer composition comprises two or more demineralizing agents. In some embodiments, the hair demineralizer composition comprises three or more demineralizing agents. In some embodiments, the hair demineralizer composition comprises four or more demineralizing agents. In some embodiments, the hair demineralizer composition comprises five or more demineralizing agents. In some embodiments, the hair demineralizer composition comprises one or more antioxidants. In some embodiments, the hair demineralizer composition comprises two or more antioxidants. In some embodiments, the hair demineralizer composition comprises three or more antioxidants.

In some embodiments, the hair demineralizer composition comprises a pH of about 6 to about 8. In some embodiments, the hair demineralizer composition comprises a pH of about 7 to about 9. In some embodiments, the hair demineralizer composition comprises a pH of about 7 to about 8.

In some embodiments, the hair demineralizer composition comprises one or more anti-redeposition agents. The anti-redeposition agent may interact with a metal ion or a metal ion complexed with a demineralizing agent. In some embodiments, the anti-redeposition agent prevents precipitation of the metal ion or of the metal ion complexed with a demineralizing agent. In some embodiments, the anti-redeposition agent comprises a plurality of negative charges. In some instances, the plurality of negative charges interacts with the metal ion or the metal ion complexed with the demineralizing agent.

In some embodiments, the hair demineralizer composition of the present disclosure is selected from Table 5, Table, 6 Table 7, Table 8, or Table 10A.

### Demineralizing Agents

In some embodiments, demineralizing agents described herein target and bind with the primary metals found in hard water. Primary metals directly impact the hair fiber texture, appearance, and structural integrity. Additionally, primary metals limit the ability of various molecules and compounds to penetrate the hair to resulting in the reduction of the efficacy of certain hair treatments. In some embodiments, the primary metals comprise calcium (Ca) and magnesium (Mg). Demineralizing agents described herein may also target and bind with secondary metals (trace metals) found in hard water. These secondary metals increase oxidation reactions at the surface and interior of the hair which may alter chemical treatments and increase the potential for damage to the hair during chemical treatments. In some embodiments, the secondary metals comprise copper (Cu), iron (Fe), chromium (Cr), nickel (Ni), aluminum (Al), lead (Pb), zinc (Zn), cadmium (Cd), mercury (Hg), arsenic (As), barium (Ba), cobalt (Co), manganese (Mn), tin (Sn), tungsten (W), antimony (Sb), beryllium (Be), boron (B), bismuth (Bi), cesium (Cs), lithium (Li), molybdenum (Mo), selenium (Se), strontium (Sr), thallium (Ti), titanium (Ti), vanadium (V), scandium (Se), gallium (Ga), yttrium (Y), niobium (Nb), technetium (Tc), ruthenium (Ru), rhodium (Rh), palladium (Pd), indium (In), tellurium (Te), rhenium (Re), osmium (Os), or iridium (ir), or combinations of two or more thereof.

The demineralizing agents disclosed herein comprise chelating agents. The term “chelating agents” refers to chemical compounds that form a stable, water-soluble complex by binding tightly to metal ions. They are also known as chelants or chelators, and these terms are used interchangeably herein. In some embodiments, the demineralizing agents comprises chelating agents. In some embodiments, the chelating agents bind metal ions by forming a molecular cage around the metal ions. In sonic embodiments, the chelating agent forms at least two bonds with the metal or metal ion. In some embodiments, the chelating agent coordinates to at least two sites on a metal or metal ion. In some embodiments, the demineralizing agent comprises a sequestrant. In some embodiments, the sequestrant forms at least one bond with the metal or metal ion. In some embodiments, the sequestrant coordinates to at least one site on a metal or metal ion.

In some embodiments, the demineralizing agents bind irreversibly with metal ions. In some embodiments, the one or more demineralizing agent binds to a metal ion that is described by an affinity constant, such as the dissociation constant (K_{D}). The dissociation constant is defined as the ratio of the product of the unbound chelator and unbound metal ion to the complex chelator and metal ion, and is expressed in units of concentration, such as moislliter (L). In some embodiments, the chelator binds to a metal ion irreversibly, and the K_{D} of the reaction may be less than about 1×10⁻⁷ M. In some instances, the chelator binds to the metal ion with a K_{D} of about 10×10⁻⁹ M, about 9×10⁻⁹ M, about 8×10⁻⁹ M, about 7×10⁻⁹ M, about 6×10⁻⁹ M, about 5×10⁻⁹ M about 4×10⁻⁹ M, about 3×10⁻⁹ M about 2×10⁻⁹ M, about 1×10⁻⁹ M, 10×10⁻⁸ M, about 9×10⁻⁸ M, about 8×10⁻⁸ M, about 7×10⁻⁸ M, about 6×10⁻⁸ M, about 5×10⁻⁸ M, about 4×10⁻⁸ M, about 3×10⁻⁸ M, about 2×10⁻⁸ M, about 1×10⁻⁸ M, 10×10⁻⁷ M, about 9×10⁻⁷ M, about 8×10⁻⁷ M, about 7×10⁻⁷ M, about 6×10⁻⁷ M, about 5×10⁻⁷ M, about 4×10⁻⁷ M, about 3×10⁻⁷ M, about 2×10⁻⁷ M, about 1×10⁻⁷ M, 10×10⁻⁶ M, about 9×10⁻⁶ M, about 8×10⁻⁶M, about 7×10⁻⁶ M, about 6×10⁻⁶ M, about 5×10⁻⁶ M, about 4×10⁻⁶ M, about 3×10⁻⁶ M, about 2×10⁻⁶ M, about 1×10⁻⁶ M, 10×10⁻⁵ M, about 9×10⁻⁵ M, about 8×10⁻⁵ M, about 7×10⁻⁵ M, about 6×10⁻⁵ M, about 5×10⁻⁵ M, about 4×10⁻⁵ M, about 3×10⁻⁵ M, about 2×10⁻⁵ M, or about 1×10⁻⁵ M.

In some embodiments, the hair demineralizer composition comprises one or more demineralizing agents. In some embodiments, the hair demineralizer composition comprises at least one demineralizing agent. In some embodiments, the hair demineralizer composition comprises at least two demineralizing agents. In some embodiments, the hair dernineralizer composition comprises at least three demineralizing agents. In some embodiments, the hair demineralizer composition comprises at least four demineralizing agents. In some embodiments, the hair demineralizer composition comprises at least five demineralizing agents. In some embodiments, the hair demineralizer composition comprises at least six demineralizing agents. In some embodiments, the hair dernineralizer composition comprises at least seven demineralizing, agents. In some embodiments, the hair demineralizer composition comprises at least eight demineralizing agents. In some embodiments, the hair demineralizer composition comprises at least nine demineralizing agents. In some embodiments, the hair demineralizer composition described herein comprises a plurality of chelating agents that work synergistically to prevent and remove mineral buildup accumulated in the hair.

In some embodiments, the one or more demineralizing agents comprises tetrasodium ethylenediaminetetraacetic acid (EDTA). In some embodiments, the one or more demineralizing agents comprises tetrasodium ethylenediaminetetraacetic acid (EDTA), sodium phytate, phytic acid, or a combination thereof. In some embodiments, the one or more demineralizing agents comprises tetrasodium ethylenediaminetetraacetic acid (EDTA), sodium phytate, phytic acid, sodium gluconate, or a combination thereof. In some embodiments, the one or more demineralizing agents comprises tetrasodium ethylenediaminetetraacetic acid (EDTA), trisodium dicarboxymethyl alaninate (MGDA), tetrasodium glutamate diacetate (GLDA), or a combination thereof. In some embodiments, the one or more demineralizing agents comprises tetrasodium EDTA, sodium phytate, phytic acid, sodium gluconate, trisodium dicarboxymethyl alaninate (MGDA), or a combination thereof. In some embodiments, the one or more demineralizing agents comprises sodium phytate, sodium gluconate, trisodium dicarboxymethyl alaninate (MGDA), or a combination thereof. In some embodiments, the one or more demineralizing agents comprises tetrasodium EDTA, sodium gluconate, trisodium dicarboxymethyl alaninate (MGDA), tetrasodium GLDA, or a combination thereof. In some embodiments, the one or more demineralizing agents comprises tetrasodium EDTA, sodium phytate, sodium gluconate, trisodium dicarboxymethyl alaninate (MGDA), tetrasodium GLDA, or a combination thereof. In some embodiments, the one or more demineralizing agents comprises tetrasodium EDTA, sodium phytate, phytic acid, sodium gluconate, trisodium dicarboxymethyl alaninate (MGDA), tetrasodium GLDA, or a combination thereof. In some embodiments, the one or more demineralizing agents comprises tetrasodium EDTA, sodium phytate, phytic acid, sodium gluconate, trisodium dicarboxymethyl alaninate (MGDA), tetrasodium GLDA, or a combination thereof. Non-limiting examples of the chelating agents include trisodium dicarboxymethyl alaninate (MGDA), sodium phytate, phytic acid, sodium gluconate, tetrasodium glutamate diacetate (GI-DA), tetrasodium ethylenediaminetetraacetic add (EDTA), trisodium EDTA, disodium EDTA, pentasodium pentetate, trisodium ethylenediamine succinate, sodium thiosulfate, caprylhydroxamic acid, diiospropyl oxalate, disodium EDIA-copper, hydroxyethylethylenediaminetriacetic acid (HEDTA), oxalic acid, potassium citrate, sodium citrate, sodium oxalate, tris(2-hydroxyethypammonium trihydrogen ethylenediaminetetraaceta e (TEA-EDTA), trisodium FIEDTA, or combinations of two or more thereof.

In some embodiments, the hair demineralizer composition comprises one or more demineralizing agents. In some embodiments, the hair demineralizer composition comprises at least 2 demineralizing agents. In some embodiments, the hair demineralizer composition comprises at least 3 demineralizing agents. In some embodiments, the hair demineralizer composition comprises at least 4 demineralizing, agents. In some embodiments, the hair demineralizer composition comprises at least 5 demineralizing agents.

In some embodiments, the one or more demineralizing agents comprises a chelator, a sequestrant, or a combination thereof. In some embodiments, the one or more demineralizing agents is a chelator or sequestrant

In some embodiments, the one or more demineralizing agents comprises at least two demineralizing agents. In some embodiments, the one or more demineralizing agent comprises trisodium dicarboxymethyl alaninate (MGDA), sodium phytate, phytic acid, sodium gluconate, tetrasodium glutamate diacetate (GLDA), tetrasodium ethylenediaminetetraacetic acid (EDTA), trisodium EDTA, disodium EDTA, pentasodium pentetate, trisodium ethylenediamine succinate, sodium thiosulfate, caprylhydroxamic acid, diiospropyl oxalate, disodium EDTA-copper, Flydroxyethylethylenediaminetriacetic acid (ELEDTA), oxalic acid, potassium citrate, sodium citrate, sodium oxalate, tris(2-hydroxyethypammonium trihydrogen ethylenediaminetetraacetate (TEA-EDTA), or trisodium HEDTA, or combinations of two or more thereof. In some embodiments, the one or more demineralizing agents comprises trisodium MGDA and sodium phytate. In some embodiments, the one or more demineralizing agents comprises trisodium MGDA and phytic acid. In some embodiments, the one or more demineralizing agents comprises trisodium MGDA and sodium gluconate. In some embodiments, the one or more demineralizing agents comprises tri sodium MGDA and tetrasodium GLDA. In some embodiments, the one or more demineralizing agents comprises trisodium MGDA and one of tetrasodium GLDA, tetrasodium EDTA, trisodium EDTA, or disodium EDTA. In some embodiments, the one or more demineralizing agents comprises trisodium MGDA and tetrasodium EDTA. In some embodiments, the one or more demineralizing agents comprises trisodium MGDA and pentasodium pentetate. In some embodiments, the one or more demineralizing agents comprises tetra.sodium EDTA and tetrasodium GLDA. In some embodiments, the one or more demineralizing agents comprises di sodium EDTA and tetrasodium EDTA. In some embodiments, the one or more demineralizing agents comprises disodium EDTA and trisodium ethylenediamine succinate. In some embodiments, the one or more demineralizing agents comprises trisodium MGDA and sodium thiosulfate. In some embodiments, the one or more demineralizing agents comprises trisodium MGDA and caprylhydroxamic acid.

In some embodiments, the one or more demineralizing, agents comprises at least three demineralizing agents. In sonic embodiments, the at least three demineralizing agents comprises trisodium MGDA and at least two demineralizers comprising sodium phytate, phytic acid, sodium gluconate, tetrasodium glutamate diacetate (GLDA), tetrasodium ethylenediaminetetraacetic acid (EDTA), trisodium EDTA, disodium EDTA, trisodium ethylenediamine succinate, sodium thiosulfate, caprylhydroxamic acid, diiospropyl oxalate, disodium EDTA-copper, pentasodium pentetate, hydroxyethylethylenediaminetriacelic acid (HEDTA), oxalic acid, potassium citrate, sodium citrate, sodium oxalate, tris(2-hydroxyethyl)ammonium trihydrogen ethylenedia.minetetraacetate (TEA-EDTA), or trisodium HEDTA, or a combination of two or more thereof. In some embodiments, the at least three demineralizing agents comprises pentasodium pentetate, trisodium MGDA, and sodium phytate. In some embodiments, the at least three demineralizing agents comprises tetrasodium GLDA, tetrasodium EDTA, or oxalic acid. In some embodiments, the at least three demineralizing agents comprises trisodium MGDA, sodium phytate, and sodium gluconate. In some embodiments, the at least three demineralizing agents comprises sodium phytate, sodium gluconate, and tetrasodium GLDA. In some embodiments, the at least three demineralizing agents cotnprises sodium gluconate, tetrasodium GLDA, and tetrasodium EDTA. In some embodiments, the at least three demineralizing agents comprises sodium gluconate, tetrasodium GLDA, and tetrasodium EDTA. In some embodiments, the at least three demineralizing agents comprises trisodium MGDA, tetrasodium GLDA, and tetrasodiurn EDTA. In some embodiments, the at least three demineralizing agents comprises trisodium MGDA, sodium gluconate, and tetrasodium EDTA. In some embodiments, the at least three demineralizing agents comprises trisodium MGDA, sodium gluconate, and tetrasodium GLDA. In sonic embodiments, the at least three demineralizing agents comprises trisodium MGDA, sodium gluconate, and potassium citrate. In some embodiments, the at least three demineralizing agents comprises sodium thiosulfate, sodium gluconate, and potassium citrate. In some embodiments, the at least three demineralizing agents capryihydroxamic acid, sodium gluconate, and potassium citrate. In some embodiments, the at least three demineralizing agents comprises sodium citrate, sodium gluconate, and potassium citrate. In some embodiments, the at least three demineralizing agents comprises trisodium HEDTA, sodium gluconate, and potassium citrate. In some embodiments, the at least three demineralizing agents comprises sodium thiosulfate, capryihydroxamic acid, and diiospropyl oxalate, in some embodiments, the at least three demineralizing agents comprises trisodium dicarboxymethyl alaninate (MGDA), sodium phytate, phytic acid, sodium gluconate, tetrasodium glutamate diacetate (GLDA), tetrasodium ethylenediaminetetraacetic acid (EDTA), trisodium EDTA, disodium EDTA, pentasodium pentetate, trisodium ethylenediamine succinate, sodium thiosulfate, capryihydroxamic acid, diiospropyl oxalate, disodium EDTA-copper, hydroxyethylethylenediaminetriacetic acid (HEDTA), oxalic acid, potassium citrate, sodium citrate, sodium oxalate, tris(2-hydroxyethyl)ammonium trihydrogen ethylenediaminetetraacetate (TEA-EDTA), or trisodium HEDTA, or a combination of three or more thereof.

In some embodiments, the one or more demineralizing agents comprises at least four demineralizing agents. In some embodiments, the at least four demineralizing agents comprises trisodium MGDA, sodium phytate, phytic acid, and sodium gluconate. In some embodiments, the at least four deminerali zing agents comprises trisodium MGDA, sodium phytate, sodium gluconate, and tetrasodium GLDA. In some embodiments, the at least four demineralizing agents comprises id sodium MGDA, sodium gluconate, tetrasodium GLDA, and tetrasodium EDTA. In some embodiments, the at least four demineralizing agents comprises sodium phytate, sodium gluconate, tetrasodiurn GLDA, and tetrasodium EDTA. In some embodiments, the at least four demineralizing agents comprises sodium phytate, sodium phytate, tetra.sodium GLDA, and tetra.sodium EDTA. In some embodiments, the at least four demineralizing agents comprises sodium phytate, sodium phytate, tetrasodium GLIDA, and tetrasodium EDTA. In some embodiments, the at least four demineralizing agents comprises tetrasodium EDTA, trisodium EDTA, disodium EDTA, and sodium gluconate. In some embodiments, the at least four demineralizing agents comprises pentasodium pentetate, tetrasodium GLDA, trisodium MGDA, and sodium phytate. In some embodiments, the at least four demineralizing agents comprises trisodium ethylenediamine succinate, sodium thiosulfate, and diisopropyl oxalate. In some embodiments, the at least four demineralizing agents comprises disodium EDTA-copper, oxalic acid, trisodium HEDTA, and sodium citrate. In some embodiments, the at least four demineralizing agents comprises oxalic acid, trisodium HEDTA, sodium citrate, and caprylhydroxamic acid. In some embodiments, the at least four demineralizing agents comprises oxalic acid, trisodium HEDTA, sodium citrate, and diisopropyl oxalate. In some embodiments, the at least four deminerali zing agents comprises trisodium dicarboxymethyl alaninate (MGDA), sodium phytate, phytic acid, sodium gluconate, tetra.sodium glutamate diacetate (GLDA), tetrasodium ethylenediaminetetraacetic acid (EDTA), trisodium EDTA, disodium EDTA, pentasodium pentetate, trisodium ethylenediamine succinate, sodium thiosulfate, caprylhydroxamic acid, diiospropyl oxalate, disodium EDTA-copper, Hydroxyethylethylenediaminetriacetic acid (HEDTA), oxalic acid, potassium citrate, sodium citrate, sodium oxalate, tris(2-hydroxyethyl)ammonium trihydrogen ethylenediaminetetraacetate (TEA-EDTA), or tri sodium HEDTA, or a combination of four or more thereof.

In some embodiments, the one or more demineralizing, agents comprises at least five demineralizing agents. In some embodiments, the at least five demineralizing agents comprises trisodium MGDA, sodium phytate, sodium gluconate, tetrasodium GLDA, and tetrasodium EDTA. In some embodiments, the at least five demineralizing agents comprises sodium phytate, phytic acid, sodium gluconate, tetrasodium GLDA, and tetrasodium EDTA. In some embodiments, the at least five demineralizing agents comprises sodium phytate, phytic acid, sodium gluconate, tetrasodium GLDA, and sodium citrate. In some embodiments, the at least five demineralizing agents comprises trisodium MGDA, sodium phytate, sodium gluconate, tetrasodium GLDA, and sodium citrate. In some embodiments, the at least five demineralizing agents comprises trisodium MGDA, sodium citrate, sodium gluconate, tetrasodium GLDA, and tetrasodium EDTA. In some embodiments, the at least five demineralizing agents comprises sodium citrate, sodium gluconate, tetrasodium GLDA, caprylhydroxamic acid, and diisopropyl oxalate. In some embodiments, the at least five demineralizing agents comprises sodium citrate, potassium citrate, sodium gluconate, tetrasodium GLDA, and diisopropyl oxalate. In some embodiments, the at least five demineralizing agents comprises tetrasodium EDTA, sodium gluconate, tetrasodium GLDA, caprylhydroxamic acid, and diisopropyl oxalate. In some embodiments, the at least five demineralizing agents comprises tetrasodium EDTA, potassium citrate, sodium gluconate, tetrasodium GLDA, and diisopropyl oxalate. In some embodiments, the at least five demineralizing agents comprises trisodium MGDA, sodium phytate, phytic acid, sodium gluconate, tetrasodium glutamate diacetate, tetrasodium EDTA, or a combination of five or more thereof. In some embodiments, the at least five demineralizing agents comprises trisodium dicarboxymethyl alaninate (MGDA), sodium phytate, phytic acid, sodium gluconate, tetrasodium glutamate diacetate (GLDA), tetrasodium ethylenediaminetetraacetic acid (EDTA), trisodium EDTA, disodium EDTA, pentasodium pentetate, trisodium ethylenediamine succinate, sodium thiosulfate, caprylhydroxamic acid, diiospropyl oxalate, disodium EDTA-copper, Hydroxyethylethylenediaminetriacetic acid (HEDTA), oxalic acid, potassium citrate, sodium citrate, sodium oxalate, tris(2-hydroxyethyl)ammonium trihydrogen ethylenediaminetetraacetate (TEA-EDTA), or trisodium HEDTA, or a combination of five or more thereof.

In some embodiments, the one or more demineralizing agents is chosen based on the ability of the one or more demineralizing agents to complex with a metal. In some embodiments, the one or more demineralizing agents is chosen based on the ability of the one or more demineralizing agents to complex with a primary metal, a secondary metal, or a combination thereof. In some embodiments, the one or more demineralizing agents is selected based on the ability to complex with a primary metal. In some embodiments, the one or more demineralizing agents complexes with Ca, Mg, or a combination thereof. In some embodiments, the one or more demineralizing agents is selected based on the ability to complex with a secondary metal. In some embodiments, the one or more demineralizing agents complexes with Cu, Fe, Cr, Ni, Al, Pb, Zn, Cd, Hg, As, Ba, Co, Mn, Sn, W, Sb, Be, B, Bi, Cs, Li, Mo, Se, Sr, Tl, Ti, Sc, Y, Nb, Tc, Ru, Rh, Pd, In, Te, Re, Os, or Ir, or a combination of two or more thereof. In some embodiments, the one or more demineralizing agents complexes with Ca, Mg, Cu, Fe, Cr, Ni, Al, Pb, Zn, Cd, Hg, As, Ba, Co. Mn, Sn, W, Sb, Be, B, Bi, Cs, Li, Mo, Se, Sr, TI, Ti, V, Sc, Ga, Y, Nb, Tc, Ru, Rh, Pd, In, Te, Re, Os, or Ir, or a combination of two or more thereof. In some embodiments, the one or more demineralizing agents complexes with Ca, Mg, Cu, Fe, Cr, Ni, Al, Pb, Zn, Cd, Hg, As, Ba, Co, Mn, Sn, W, Sb, Be, B, Bi, Cs, Li, Mo, Se, Sr, Tl, Ti, V, Sc, Ga, Y, Nb, Tc, Ru, Rh, Pd, In, Te, Re, Os, or Ir, or a combination of two or more thereof. In some embodiments, the one or more demineralizing agents complexes with Ca, Mg, Cu, Cr, Fe, Al, Zn, Ni Cd, Ph, or Hg, or a combination of two or more thereof. In some embodiments, the one or more demineralizing agents complexes with Ca, Mg, Cu, Cr, Fe, Al, Zn, Ni, Cd, or Pb, or a combination of two or more thereof. In some embodiments, the one or more demineralizing agents complexes with Ca, Mg, Cu, Fe, Mn, or Zn, or a combination or two or more thereof. In some embodiments, the one or more demineralizing agents complexes with Ca, Mg, Cu, Fe, or Mn, or a combination of two or more thereof. In some embodiments, the one or more demineralizing agents complexes with Ca, Mg, Fe, or Zn, or a combination of two or more thereof. In some embodiments, the one or more demineralizing agents complexes with Ca, Fe, Co, or Zn, or a combination or two or more thereof. In some embodiments, the one or more demineralizing agents complexes with Na, Cu, Li, or a combination of two or more thereof.

In some embodiments, the one or more demineralizing agents comprises trisodium MGDA, which complexes with polyvalent metal ions. For example, trisodium MGDA complexes with Ca²⁺, Cu²⁺, Fe³⁺, Mg²⁺, or Mn²⁺, or combinations of two or more thereof. In some embodiments, the one or more demineralizing agents comprises sodium phytate. For example, sodium phytate complexes with Ca²⁺, Mg²⁺, Fe³⁺, or Zn²⁺, or a combination or two or more thereof. In some embodiments, the one or more demineralizing agents comprises sodium gluconate. For example, sodium gluconate coordinates or chelates to a monovalent ion. The monovalent ion may be Na⁺, Cu⁺, or a combination or two or more thereof. In some embodiments, the one or more demineralizing agents comprises tetrasodium glutamate diacetate. For example, tetrasodium glutamate diacetate complexes with Ca²⁺, Cu²⁺, Fe³⁺, Mg²⁺, Mn²⁺, or Zn²⁺, or a combination or two or more thereof. In some embodiments, the one or more demineralizing agents comprises tetrasodium EDTA. For example, tetrasodium EDTA complexes with Fe³⁺, Co²⁺, or Ca²⁺, or a combination or two or more thereof. In some embodiments, the one or more demineralizing agents comprises sodium citrate or potassium citrate. For example, potassium citrate or sodium citrate complexes with Fe²⁺, Fe³⁺, Mn²⁺, Co²⁺, or Al³⁺, or a combination of two or more thereof. In some embodiments, the one or more demineralizing, agents comprises pentasodium pentetate. For example, pentasodium pentetate binds to Tc⁴⁺, Zn²⁺, Mg²⁺, Mn²⁺, or a combination of two or more thereof.

In some embodiments, the one or more demineralizing agents may carry a net negative charge. In some embodiments the one or more demineralizing agents may carry a total net negative charge corresponding to a sum of a net negative charge of each of the one or more demineralizing agents. In some embodiments, the one or more demineralizing agents may carry a net negative charge over a pH of about pH 6 to about pH 9, about pH 7 to about pH 8, about pH 7 to about pH 9, or of about pH 6 to about pH 8. In some embodiments, the net negative charge of each of the one or more demineralizing agents over the pH range is −1, −2, −3,−4, or −5.

In some embodiments, the total concentration (%w/w) of the one or more demineralizing agents in the hair demineralizer composition is about 0.1% to about 15%. In some embodiments, the concentration (%w/w) of each of the one or more demineralizing agents in the hair demineralizer composition is about 0.05%, about 0.1%, about 0.2%, about 0.4%, about 0.6%, about 0.8%, about 1%, about 1.2%, about 1.4%, about 1.6%, about 1.8%, about 2%, about 3%, about 4%, about 5%, about 6%, about 7%, about 8%, about 9%, about 10%, about 11%, about 12%, about 13%, about 14%, about 15%, or any values therebetween. In some embodiments, the total concentration (%w/w) of the one or more demineralizing agents in the hair demineralizer composition is about 0.1% to about 15%, about 0.2% to about 14%, about 0.4% to about 13%, about 0.6% to about 12%, about 0.8% to about 11%, about 1% to about 10%, about 1.2% to about 9%, about 1.4% to about 8%, about 1.6% to about 7%, about 1.8% and about 6%, about 2% to about 5%, or about 3% to about 4%. In some embodiments, when two or more demineralizing agents are present, the concentration of each demineralizing agent may be the same. In some embodiments, when two or more demineralizing agents are present, the concentration of each demineralizing agent may be different.

In some embodiments, the one or more demineralizing agents comprises trisodium MGDA, wherein the trisodium MGDA is present in the composition at about 0.005% to about 5% by weight, about 0.1% to about 2% by weight, about 0.5% to about 2% by weight, about 0.6% to about 2% by weight, about 0.7% to about 2% by weight, about 0.8% to about 2% by weight, about 0.9% to about 2% by weight, about 1% to about 2% by weight, about 1% to about 2% by weight, about 1.1% to about 2% by weight, about 1.2% to about 2% by weight, about 1.3% to about 2% by weight, about 1.4% to about 2% by weight, about 1.5% to about 2% by weight, 0.5% to about 1% by weight, about 0.6% to about 1% by weight, about 0.7% to about 1% by weight, about 0.8% to about 1% by weight, or about 0.9% to about 1% by weight. For example, the trisodium MGDA is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

In some embodiments, the one or more demineralizing agents comprises sodium phytate, wherein the sodium phytate is present in the composition at about 0.005% to about 5% by weight, about 0.1% to about 2% by weight, about 0.5% to about 2% by weight, about 0.6% to about 2% by weight, about 0.7% to about 2% by weight, about 0.8% to about 2% by weight, about 0.9% to about 2% by weight, about 1% to about 2% by weight, about 1% to about 2% by weight, about 1.1% to about 2% by weight, about 1.2% to about 2% by weight, about 1.3% to about 2% by weight, about 1.4% to about 2% by weight, about 1.5% to about 2% by weight, 0.5% to about 1% by weight, about 0.6% to about 1% by weight, about 0.7% to about 1% by weight, about 0.8% to about 1% by weight, or about 0.9% to about 1% by weight. For example, the sodium phytate is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

In some embodiments, the one or more demineralizing, agents comprises sodium gluconate, wherein the sodium gluconate is present in the composition at about 0.005% to about 5% by weight, about 0.1% to about 2% by weight, about 0.5% to about 2% by weight, about 0.6% to about 2% by weight, about 0.7% to about 2% by weight, about 0.8% to about 2% by weight, about 0.9% to about 2% by weight, about 1% to about 2% by weight, about 1% to about 2% by weight, about 1.1% to about 2% by weight, about 1.2% to about 2% by weight, about 1.3% to about 2% by weight, about 1.4% to about 2% by weight, about 1.5% to about 2% by weight, 0.5% to about 1% by weight, about 0.6% to about 1% by weight, about 0.7% to about 1% by weight, about 0.8% to about 1% by weight, or about 0.9% to about 1% by weight. For example, the sodium gluconate is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

In some embodiments, the one or more demineralizing agents comprises tetrasodium glutamate diacetate, wherein the tetrasodium glutamate diacetate is present in the composition at about 0.005% to about 5% by weight, about 0.1% to about 2% by weight, about 0.5% to about 2% by weight, about 0.6% to about 2% by weight, about 0.7% to about 2% by weight, about 0.8% to about 2% by weight, about 0.9% to about 2% by weight, about 1% to about 2% by weight, about 1% to about 2% by weight, about 1.1% to about 2% by weight, about 1.2% to about 2% by weight, about 1.3% to about 2% by weight, about 1.4% to about 2% by weight, about 1.5% to about 2% by weight, 0.5% to about by weight, about 0.6% to about 1% by weight, about 0.7% to about 1% by weight, about 0.8% to about 1% by weight, or about 0.9% to about 1% by weight, For example, the tetrasodium glutamate diacetate is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

In some embodiments, the one or more demineralizing, agents comprises tetrasodium EDTA, wherein the tetrasodium EDTA is present in the composition at about 0.005% to about 5% by weight, about 0.1% to about 2% by weight, about 0.5% to about 2% by weight, about 0.6% to about 2% by weight, about 0.7% to about 2% by weight, about 0.8% to about 2% by weight, about 0.9% to about 2% by weight, about 1% to about 2% by weight, about 1% to about 2% by weight, about 1.1% to about 2% by weight, about 1.2% to about 2% by weight, about 1.3% to about 2% by weight, about 1.4% to about 2% by weight, about 1.5% to about 2% by weight, 0.5% to about 1% by weight, about 0.6% to about 1% by weight, about 0.7% to about 1% by weight, about 0.8% to about 1% by weight, or about 0.9% to about 1% by weight. For example, the tetrasodium EDTA is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

### Antioxidants

Antioxidants are synthetic or naturally-occurring molecules that inhibit oxidation reactions by neutralizing unpaired electrons of free radicals through a resonance-stabilized bonding network. They act as reactive oxygen species (ROS) scavengers to reduce oxidative damage. Some non-limiting examples of antioxidants include vitamin C (ascorbic acid) and vitamin E (alpha-tocopherol). In some embodiments, the one or more antioxidants reduce oxidation reactions on hair that are caused by secondary (trace) metals, which may remain after removal by the demineralizing agents. Most antioxidants are effective to reduce oxidation reactions under acidic conditions. By contrast, in some embodiments, the one or more antioxidants work effectively under alkaline conditions to match an optimal activity of the one or more demineralizing agents. In some embodiments, the optimal activity of the one or more demineralizing agents comprises binding to at least about 50%, to at least about 60%, at least about 70%, at least about 80%, at least about 90%, or at least about 95% of metal ions. In some embodiments, the one or more antioxidants of the hair demineralizer composition of the present disclosure may be effective at neutral or alkaline pH ranges. In some embodiments, the one or more antioxidants of the hair demineralizer composition of the present disclosure may be effective at pH of about 6 to about 9, for example, at pH of about 7 to about 8.

In some embodiments, the hair demineralizer composition comprises one or more antioxidants. In some embodiments, the antioxidant comprises a peptide-derived antioxidant, In some embodiments, the peptide-derived antioxidant comprises phenylalanine, valine, cysteine, alanine, histidine, or a combination thereof. In some embodiments, the antioxidants comprise dipeptides of alanine and histidine. In some embodiments, the peptide-derived antioxidant comprises carnosine, valine-cysteine, di-proline, di-phenylalanine, or a combination thereof. In some embodiments, the peptide-derived antioxidant comprises camosine, In some embodiments, the antioxidant comprises carnosine, ascorbic acid, tetrahexyldecyl ascorbate, bioflavonoids, lycopene, Danens carota saliva root cell culture lysate, monoammonium glycyrrhizinate, alpha lipoic acid (thioctic acid), Leontopodium alpinum (edelweiss) extract, Rosmarinus officinalis (rosemary) leaf extract, Vitis vinifera (grape) seed extract, Camellia sinensis leaf extract, Quercus robur wood extract, hydrolyzed olive fruit, Olea europea (olive) leaf extract, oleuropin, Punica granatum bark/fruit extract, punicalagin, ellagic acid, polyphenols, epigallocatechin (EGCG), tannins, sulforaphane, resveratrol, nordihydroguaiaretic acid, or thioctic acid, or combinations of two or more thereof.

In some embodiments, the total concentration (%w/w) of the one or more antioxidants in the hair demineralizer composition is about 0.01% to about 5%. In some embodiments, the concentration (%w/w) of each of the one or more antioxidants in the hair demineralizer composition is about 0.01%, about 0.02%, about 0.04%, about 0.06%, about 0.08%, about 0.1%, about 0.5%, about 1%, about 1.5%, about 2%, about 2.5%, about 3%, about 3.5%, about 4%, about 4.5%, about 5%, or any values therebetween. In some embodiments, the concentration (%w/w) of each of the one or more antioxidants in the hair demineralizer composition is about 0.01% to about 5%, about 0.02% to about 4.5%, about 0.04% to about 4%, about 0.06% to about 3.5%, about 0.08% to about 3%, about 0.1% to about 2.5%, about 0.5% to about 2%, or about 1% to about 5%.

In some embodiments, the demineralizer composition comprises carnosine at about 0.005% to about 5% by weight, about 0.1% to about 5% by weight, about 0.5% to about 5% by weight, about 1% to about 5% by weight, about 2% to about 5% by weight, about 1% to about 2% by weight. For example, the carnosine is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

### Anti-Redeposition Agent

The hair demineralizer composition may comprise one or more anti-redeposition agents. In some embodiments, the anti-redeposition agents disclosed herein are rheology modifiers or biodegradable functionalized biopolymers. In some embodiments, the anti-redeposition agents have a negative charge similar to the hair or chelating agents. In some embodiments, the anti-redeposition agents play a role in the demineralizing capacity of the hair demineralizer composition. In some embodiments, the anti-redeposition agents inhibit calcium carbonate (CaCO₃) precipitation. In some embodiments, the anti-redeposition agents keep metal ions dispersed in water, so the metal ions do not re-deposit on hair. In some embodiments, the anti-redeposition agent prevents metal ions, metal/chelator complexes, or a combination thereof. In some embodiments, the anti-redeposition agent comprises a plurality of negatively charged groups. In some embodiments, the plurality of negatively charged groups coordinates to a metal or metal ion. In some embodiments, each of the plurality of negatively charged groups coordinates to one metal or metal ion. In some embodiments, at least two negatively charged groups coordinates to one metal or metal ion. In some embodiments, each of the plurality of negatively charged groups coordinates to at least one metal or metal ion. In some embodiments, the hair demineralizer composition comprises one or more anti-redeposition agents selected from the group consisting of: sodium carboxymethyl inulin, xanthan gum, hydroxyethylcellutose, hydroxymethylcellulose, cellulose, cellulose gum, microcrystalline cellulose, carboxymethylcellulose, pullulan, sclerotium gum, tetragonoloba (guar) gum, acacia senegal gum, hydroxypropyl starch phosphate, lithium magnesium sodium silicate, or sodium magnesium fluorosilicate, or a combination of two or more thereof. In some embodiments, the anti-redeposition agents comprise sodium carboxymethyl inulin.

In some embodiments, the total concentration (%w/w) of the one or more anti-redeposition agents in the hair demineralizer composition is about 0.01% to about 10%. In some embodiments, the concentration (%w/w) of each of the one or more anti-redeposition agents in the hair demineralizer composition is about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, or any values therebetween. In some embodiments, the concentration (%w/w) of the one or more anti-redeposition agents in the hair demineralizer composition is about 0.01% to about 10%, about 0.05% to about 9.5%, about 0.1% to about 9%, about 0.2% to about 8.5%, about 0.3% to about 8%, about 0.4% to about 7.5%, about 0.5% to about 7%, about 0.6% to about 6.5%, about 0.7% to about 6%, about 0.8% to about 5.5%, about 0.9% to about 5%, about 1% to about 4.5%, about 1.5% to about 4%, about 2% to about 3.5%, or about 2.5% to about 3%.

In some embodiments, the demineralizing composition comprises sodium carboxymethyl inulin present in the composition at about 0.005% to about 5% by weight, about 0.1% to about 2% by weight, about 0.5% to about 2% by weight, about 0.6% to about 2% by weight, about 0.7% to about 2% by weight, about 0.8% to about 2% by weight, about 0.9% to about 2% by weight, about 1% to about 2% by weight, about 1% to about 2% by weight, about 1.1% to about 2% by weight, about 1.2% to about 2% by weight, about 1.3% to about 2% by weight, about 1.4% to about 2% by weight, about 1.5% to about 2% by weight, 0.5% to about 1% by weight, about 0.6% to about 1% by weight, about 0.7% to about 1% by weight, about 0.8% to about 1% by weight, or about 0.9% to about 1% by weight, For example, the sodium carboxymethyl inulin is present in the composition at about 0.005%, 0.05%, 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

### Surfactant

The hair demineralizer composition may comprise one or more surfactants. In some embodiments, the surfactant is a solubilizer. In some embodiments, the solubilizer includes potysorbate 20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, or sodium suifactin, or combinations of two or more thereof. In some embodiments, the surfactant is a cleanser. In some embodiments, the surfactant is a cleanser comprising Sapindus mukorossi (soapnut) peel extract, potassium cocoyl hydrolyzed oat protein, Starmella bombicola/brassica oil ferment, sophorolipids, canola sophorolipids, rha.mnolipids, glycolipids, lauramidopropyl hydroxysultaine, lauryl hydroxysultaine, sodium lauroyl sarcosinate, sodium lauroyl isethionate, sodium cocoyl isethionate, lauroyl sarcosine, sodium lauroyl glutamate, sodium methyl cocoyl taurate, di sodium cocoamphodiacetate, sodium C₁₄₋₁₆ olefin sulfonate, sodium C₁₄₋₁₆ alpha olefin sulfonate, benzethonium chloride, cocamidopropylamine oxide, decylamine oxide, decyl glucoside, lawyl glucoside, coca-glucoside, cocamidopropyl betaine, betaine, laurvi betaine, lauramidopropyl hydroxysultaine, cocamidopropyl hydroxysuttaine, maltooligosyl glucoside, hydrogenated starch hydrolysate, or combinations thereof. In some embodiments, the cleanser comprises biosurfactant from soapnut. In some embodiments, the cleanser comprises Sapindiis mukaorossi (soapnut) extract. The soapnut extract is a source of saponins that are biodegradable, making them natural surfactant molecules with high environmental compatibility and lower toxicity. The soapnut extract is effective in hard water due to its chelating ability and good foaming ability. It can be used for gentle cleansing to help washout or rinse the chelator.

In some embodiments, the total concentration (%w/w) of the one or more surfactants in the hair demineralizer composition is about 0.05% to about 30%. In some embodiments, the concentration (%w/w) of each the one or more surfactants in the hair demineralizer composition is about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 12%, about 14%, about 16%, about 18%, about 20%, about 22%, about 24%, about 26%, about 28%, about 30%, or any values therebetween. In some embodiments, the concentration (%w/w) of the one or more surfactants in the hair demineralizer composition is about 0.05% to about 30%, about 0.1% to about 28%, about 0.5% to about 26%, about 1% to about 24%, about 1.5% to about 22%, about 2% to about 20%, about 2.5% to about 18%, about 3% to about 16%, about 3.5% to about 14%, about 4% to about 12%, about 4.5% to about 10%, about 5% to about 9.5%, about 5.5% to about 9%, about 6% to about 8.5%, about 6.5% to about 8%, or about 7% to about 7.5%.

In some embodiments, the demineralizer composition comprises polysorbate 20 at about 0% to about 5% by weight, about 0.1% to about 5% by weight, about 0.5% to about 5% by weight, about 1% to about 5% by weight, about 2% to about 5% by weight, about 1% to about 2% by weight. For example, the polysorbate 20 is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, about 2.5%, about 3%, about 3.5%, about 4%, about 4.5%, or about 5% by weight of the composition.

In some embodiments, the demineralizer composition comprises Sqpinchts mukorossi peel extract at about 0% to about 5% by weight, about 0.1% to about 5% by weight, about 0.5% to about 5% by weight, about 1% to about 5% by weight, about 2% to about 5% by weight, about 1% to about 2% by weight. For example, the Sapindus mukorossi peel extract is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, about 2.5%, about 3%, about 3.5%, about 4%, about 4.5%, or about 5% by weight of the composition.

### PH Modifiers

Primary and secondary metals are more soluble in acidic solutions. Therefore, chelating agents are typically provided in compositions having an acidic pH. However, specific chelating agents disclosed herein are identified to be more effective under alkaline conditions. Accordingly, the present disclosure provides a hair demineralizer composition that is effective at alkaline pH conditions. In some embodiments, the hair demineralizer composition comprises one or more pH modifiers to provide optimized pH conditions. In some embodiments, the hair demineralizer composition is effective under mild alkaline conditions, at pH from about 6 to about 9, or about 7 to about 8.

In some embodiments, the hair demineralizer composition has a pH of about 6, about 6.25 about 6.5, about 6.75, about 7, about 7.25, about 7.5, about 7.75, about 8, about 8.25, about 8.5, about 8.75, about 9, or any values therebetween. In some embodiments, the hair demineralizer composition has a pH of about 6 and about 9, about 6.25 and about 8.75, about 6.5 and about 8,5, about 6.75 and about 8.25, about 7 and about 8, or about 7.25 and about 7.75.

In some embodiments, one or more pH modifiers may be present to modulate the pH of the hair composition. In sonic embodiments, the pH modifier may have a pKₐ, In some embodiments, the pKₐ of the pH modifier may be substantially the same as the pH of the hair composition. In some instances, the pH modifier may comprise a pKₐ from about 6 to about 9, from about 6 to about 8, from about 6 to about 7, from about 7 to about 9, from about 7 to 8, or from about 8 to 9. In some embodiments, the pH modifier does not react with the one or more demineralizers. In some embodiments, the pH modifier does not interact with the one or more demineralizers. In some embodiments, the one or more pH modifiers comprises lactic acid, citric acid, malic acid, gluconic acid, glucuronic acid, glycolic acid, tartaric acid, azelaic acid, acetic acid, sodium hydroxide, triethanolamine, or L-arginine, or combinations of two or more thereof. In some embodiments, the one or more pH modifiers comprises lactic acid, citric acid, malic acid, giuconic acid, glucuronic acid, glycolic acid, tartaric acid, azelaic acid, acetic acid, sodium hydroxide, triethanolamine, or L-arginine, or combinations of two or more thereof.

In some embodiments, the total concentration (%w/w) of the one or more pH modifiers in the hair demineralizer composition is about 0.01% to about 5%. In some embodiments, the concentration (%w/w) of each of the one or more pH modifiers in the hair demineralizer composition is about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.5%, about 2%, about 2.5%, about 3%, about 3.5%, about 4%, about 4.5%, about 5%, or any values therebetween. In some embodiments, the concentration (%w/w) of the one or more pH modifiers in the hair demineralizer composition is about 0.01% and about 5%, about 0.05% and about 4.5%, about 0.1% and about 4%, about 0.15% and about 3.5%, about 0.2% and about 3%, about 0.25% and about 2.5%. about 0.3% and about 2%, about 0.35% and about 1.5%, about 0.4% and about 1%, about 0.45% and about 0.95%, about 0.5% and about 0.9%, about 0.55% and about 0.85%, about 0.6% and about 0.8%, about 0.6% and about 0.75%, or about 0.65% and about 0.7%.

In some embodiments, the &mineralizing. composition comprises lactic acid present in the composition at about 0% to about 2% by weight, about 0.1% to about 2% by weight, about 0.5% to about 2% by weight, about 1% to about 2% by weight, about 0.05% to about 1% by weight, about 0.1% to about 1% by weight, or about 0.5% to about 1% by weight. For example, the lactic acid is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.8%, about 1.9%, about 2%, or about 5% by weight of the composition.

### Solvents And Excipients

In some embodiments, the hair demineralizer composition comprises one or more solvents. In some embodiments, the one or more solvents comprises an aqueous solvent. In some embodiments, the one or more solvents comprises a polar solvent, a nonpolar solvent, or a combination thereof. In some embodiments, the one or more solvents comprises water, an alcohol, a glycol, an ester, an ether, a ketone, an organic acid, or a halogenated solvent, or combinations of two or more thereof. In some embodiments, the alcohol comprises methanol, ethanol, propanol, isopropanol, 1-butanol, 2-butanol, isobutanol, 1,4-butanediol, benzyl alcohol, or combinations of two or more thereof. In some embodiments, the glycol comprises glycerol, ethylene glycol, or a combination thereof. In some embodiments, the ester comprises t-butyl acetate, butyl acetate, ethylene carbonate, propyl acetate, dimethyl carbonate, ethyl acetate, ethyl propionate, methyl acetate, ethyl format, or combinations of two or more thereof. In some embodiments, the ether comprises diethylene glycol, anisole, diphenyl ether, dibutyl ether, t-butyl ethyl ether, t-amyl methyl ether, dimethyl isosorbide, ethoxybenzene, or combinations of two or more thereof. In some embodiments, the ketone comprises acetone, methylethyl ketone, cyciohexanone, cyclopentanone, or combinations of two or more thereof. In some embodiments, the organic acid comprises propionic acid, acetic anhydride, acetic acid, tri methyl citrate, triethyl citrate, or combinations of two or more thereof. In some embodiments, the halogenated solvent comprises chlorobenzene, trichloroacetonitrile, chloroacetic acid, chlorotrifluoropropene, or combinations of two or more thereof. In some embodiments, the one or more solvents comprises water and an alcohol. In some instances, the one or more solvents comprises water, methanol, or a combination thereof. In some instances, the one or more solvents comprises water, ethanol, or a combination thereof. In some embodiments, the one or more solvents comprises water, an alcohol, an ether, or combinations of two or more thereof. In some embodiments, the one or more solvents comprises water, an alcohol, an ether, an organic acid, or combinations of two or more thereof. In some embodiments, the one or more solvents comprises water, an alcohol, an ether, an organic acid, a halogenated solvent, or combinations of two or more thereof. In some embodiments, the one or more solvents comprise water, alcohols, dimethyl isosorbide, chlorotrifluoropropene, glycols, triethyl citrate, or combinations of two or more thereof. In some embodiments, the one or more solvents comprises water, alcohol, glycol, or combinations of two or more thereof.

In some embodiments, the total concentration (%w/w) of the one or more solvents or co-solvents in the hair demineralizer composition is about 5% to about 90%. In some embodiments, the concentration of each of the one or more solvents or co-solvents in the hair demineralizer composition is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or any values therebetween. In some embodiments, the concentration of the one or more solvents or co-solvents in the hair demineralizer composition is about 5% and about 90%, about 10% and about 85%, about 15% and about 80%, about 20% and about 75%, about 25% and about 70%, about 20% and about 65%, about 25% and about 60%, about 30% and about 55%, about 35% and about 50%, or about 40% and about 45%.

In some embodiments, the demineralizing composition comprises water present in the composition at about 80% to about 95% by weight, about 80% to about 85%, about 85% to about 95% by weight, about 90% to about 95% by weight, or about 85% to about 90% by weight. example, the water is present in the composition at about 80%, about 81%, about 82%, about 83%, about 84%, about 85%, about 86%, about 87%, about 88%, about 89%, about 90%, about 91%, about 92%, about 93%, about 94%, or about 95% by weight of the composition.

In some embodiments, the hair demineralizer composition comprises one or more cosmetically or dermatologically acceptable excipients. In some embodiments, the one or more cosmetically or dermatologically acceptable excipients comprises a perfume, a preservative, or a combination thereof. In some embodiments, the hair demineralizer composition further comprises one or more cosmetically or dermatologically acceptable excipients comprising a perfume, a preservative, or a combination thereof. In some embodiments, the preservative comprises gluconolactone, benzyl alcohol, calcium gluconate, sodium benzoate, phenoxyethanol, potassium sorbate, ethylhexylglycerin, caprylyl glycol, caprylhydroxamic acid, or a combination of two or more thereof.

### Methods For Demineralizing Hair

The present disclosure also provides a method for demineralizing hair using the hair demineralizer compositions disclosed herein.

In some embodiments, the method comprises contacting the hair demineralizer composition as described herein with water comprising metals, In some embodiments, the metal comprises a primary metal, a secondary metal, or a combination thereof. In some embodiments, the water is a hard water. In some embodiments, the hair demineraiizer composition comprises one or more demineralizing agents. In some embodiments, the hair demineralizer composition comprises one or more antioxidants. In some embodiments, the hair demineralizer composition has a pH of about 6 to about 9. In some embodiments, the method comprises binding of the one or more demineralizing agents to the metal. In some embodiments, each of the one or more demineralizing agents binds to a metal. In some embodiments, the binding of the one or more demineralizing agents comprises removing about 10%, about 20%, about 30%, about 40%, about 50%, about 60%, about 70%, about 80%, about 90%, about 95%, about 99%, or about 100% of each metal in the water. In some embodiments, the one or more demineralizing agents removes about 10%, about 20%, about 30%, about 40%, about 50%, about 60%, about 70%, about 80%, about 90%, about 955, about 99%, or about 100% of the total metals in the water.

In some embodiments, the method comprises contacting the hair demineralizer composition as described herein with hair comprising metals. In some embodiments, the metal comprises a primary metal, a secondary metal, or a combination thereof. In some embodiments, the water is a hard water. In sonic embodiments, the hair demineraiizer composition comprises one or more demineralizing agents. In some embodiments, the hair demineralizer composition comprises one or more antioxidants. In some embodiments, the hair demineralizer composition has a pH of about 6 to about 9. In some embodiments, the method comprises binding of the one or more demineralizing agents to the metal. In some embodiments, each of the one or more demineralizing agents binds to a metal. In some embodiments, the binding of the one or more demineralizing agents comprises removing about 10%, about 20%, about 30%, about 40%, about 50%, about 60%, about 70%, about 80%, about 90%, about 955, about 99%, or about 100% of each metal in the hair. In some embodiments, the one or more demineralizing agents removes about 10%, about 20%, about 30%, about 40%, about 50%, about 60%, about 70%, about 80%, about 90%, about 95%, about 99%, or about 100% of the total metals in the hair.

In some embodiments, the method for demineralizing hair comprises applying the demineralizer to the hair. In some embodiments, the method comprises leaving the demineralizer on the hair for a few minutes. In some embodiments, the method comprises massaging or combing the hair for even coating. In some embodiments, the method comprises washing the hair with a hair cleansing product, In some embodiments, the method comprises rinsing out the hair completely. In some embodiments, the method comprises drying the hair. In some embodiments, the method for demineralizing hair comprises applying the demineralizer to the hair, leaving the demineralizer on the hair for a few minutes, massaging or combing the hair for even coating, washing the hair with a hair cleansing product, rinsing out the hair completely, and drying the hair. In some embodiments, the method for demineralizing, hair comprises applying the demineralizer to the hair, leaving it on the hair for a few minutes, massaging or combing the hair for even coating, and continuing directly with a chemical service. In some embodiments, the method for demineralizing hair comprises applying the demineralizer to the hair, leaving it on the hair for a few minutes, adding heat, massaging or combing the hair for even coating, and either washing out or continuing with a chemical service. In some embodiments, the demineralizer composition is added directly to a cleansing product.

In some cases, after applying the demineralizer to the hair, the demineralizer sits on the hair for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, or any values therebetween. In some cases, after applying the demineralizer to the hair, the demineralizer sits on the hair for about I minute to about 30 minutes, about 2 minutes to about 29 minutes, about 3 minutes to about 28 minutes, about 4 minutes to about 27 minutes, about 5 minutes to about 26 minutes, about 6 minutes to about 25 minutes, about 7 minutes to about 24 minutes, about 8 minutes to about 23 minutes, about 9 minute to about 22 minutes, about 10 minutes to about 21 minutes, about 11 minutes to about 20 minutes, about 12 minutes to about 19 minutes, about 13 minutes to about 18 minutes, about 14 minutes to about 17 minutes, or about 15 minutes to about 16 minutes.

In some embodiments, the method comprises contacting hair with the hair demineralizer composition. In some embodiments, the method further comprises binding to about 10%, about 20%, about 30%, about 40%, about 50%, about 60%, about 70%, about 80%, about 90%, about or about 99% of a total metal ion concentration in the hair. In some embodiments, the method comprises binding to about 10%, about 20%, about 30%, about 40%, about 50%, about 60%, about 70%, about 80%, about 90%, about 95%, or about 99% of a primary metal or a secondary metal in the hair as compared to untreated hair. In some embodiments, the method comprises providing a metalldemineralizer complex, wherein the one or more demineralizing agents complexes with the metal ion to provide a metal/demineralizer complex. In some embodiments, the one or more demineralizing agents provides a metal/demineralizer complex formed from the one or more demineralizing agents complexing with about 10%, about 20%, about 30%, about 40%, about 50%, about 60%, about 70%, about 80%, about 90%, about 95%, or about 99% of the metal ions, In some embodiments, the one or more demineralizing agents complexes with about 10% to about 95%, about 20% to about 80%, about 30% to about 70%, about 40% to about 60%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 70% to about 40%, about 20% to about 50%, about 20% to about 60%, about 20% to about 70%, about 20% to about 80%, or about 20% to about 90% the metal ion present in the hair prior to contacting with the dernineralizer composition.

In some embodiments, the method further comprises removing the metal/demineralizer complex. In some embodiments, removing the melal/demineralizer complex comprises washing the hair. In some instances, washing the hair comprises washing the hair with a hair cleansing product, in some embodiments, the method comprises removing a portion of the metal ions in the hair. In some embodiments, removing a portion of the metal ions in the hair is subsequent to washing the hair. In some embodiments, removing a portion of the metal ions in the hair comprises removing about 10% to about 95%, about 20% to about 80%, about 30% to about 70%, about 40% to about 60%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, or about 20% to about 90% metal ions in the hair. In some embodiments, the amount of metal ion removed from the hair may be determined by Time-of-Flight Secondary Ion Mass Spectrometry (TOF-SIMS).

In some embodiments, after applying the demineralizer to the hair, heat may be added to increase the reaction for more severe cases of mineral buildup.

In some embodiments, the methods described herein improve hair texture and shine. In some embodiments, the methods described herein improve hair color, in some embodiments, the methods described herein prevent or improve swimmer's hair. In some embodiments, the methods described herein improve hair treatments, including perms, relaxers, bleaching, hair coloring, and the use of other hair care products such as bond builders.

### EXAMPLES

The following examples are included for illustrative purposes only and are not intended to limit the scope of the disclosure.

### Example 1

### Evaluation of specific elements on hair fibers by Time-of-Flight Secondary Ion Mass Spectrometry (TOF-SIMS) (1)

The presence of 10 elements on single hair fibers was evaluated before and after the application of an exemplary hair demineralizer disclosed herein,

A total of 3 samples were used to treat al g hair tress including Demineralizer A (trisodium MGDA, sodium phytate, sodium gluconate, tetrasodium tetrasodium EDTA; no antioxidant; pH 7-8), Demineralizer B (trisodium MGDA, sodium phytate, sodium gluconate, tetrasodium GLDA, tetrasodium EDTA; no antioxidant; pH 4-5), and a commercial product I (CP-I) (sodium gluconate and disodium EDTA). CP-I also included ascorbic acid as an antioxidant. The entire packet of CP-I was mixed with 2 oz of warm water. Bleached hair tresses were rinsed off 10 times with contaminated hard water comprising a mixture of primary and secondary metals. The primary metals include, but are not limited to, Ca and Mg. The secondary metals include, but are not limited to, iron (Fe), copper (Cu), sodium (Na), aluminum (Al), lead (Pb), cadmium (Cd), chromium (Cr), and nickel (Ni). The hair tresses were then treated with the Demineralizer A or B as disclosed herein at 40% of the w/w of the total composition. The hair tresses were massaged for 30 seconds and left for 4 minutes before rinsing with regular water.

Samples tested include i) control hair fibers treated with contaminated hard water, ii) contaminated hair fibers treated with Demineralizer A, iii) contaminated hair fibers treated with Demineralizer B, and iv) contaminated hair fibers treated with CP-I.

Elements analyzed by TOF-SIMS on hair fiber surface include iron (Fe), copper (Cu), sodium (Na), aluminum (Al), magnesium (Mg), lead (Pb), cadmium (Cd), chromium (Cr), nickel (Ni), and calcium (Ca).

TOF-SIMS makes use of the secondary ion mass spectra of atomic species or low molecular weight fragments which are formed when the sample surface is bombarded with a positively charged bismuth ion beam. Characteristic positive or negative ions, which are unique to the chemical compounds present at the point of impact of the beam, are analyzed according to the weight/charge ratio (m/z).

Three bleached hair tresses were rinsed-off 10 times with contaminated hard water. Three hair fibers were randomly selected for TOF-SIMS analysis (Control). These hair tresses were then treated with the product (Demineralizer A, Demineralizer B, and CP-I) and rinsed off with regular water. Three hair fibers were randomly selected for TOE-SIMS analysis (After treatment), The samples were measured with a TOF-SIMS system (nanoTOF II, Physical Electronics) with 30 kV Bi⁺ ions. During analysis, ow-energy electrons and Ar⁺ ions (+/−10 eV) were used to keep the sample surface charge neutral.

TOF-SIMS images were recorded on 3 single fibers from the control (Control) and the 3 treated samples (Demineralizer A, Demineralizer B, and CP-I). The distribution of the 10 target elements (Ca, Mg, Cu, Fe, Cd, Ni, Na, Al, Cr, and Pb) on the hair surface was achieved by calculating relative amounts of each as determined in the MS.

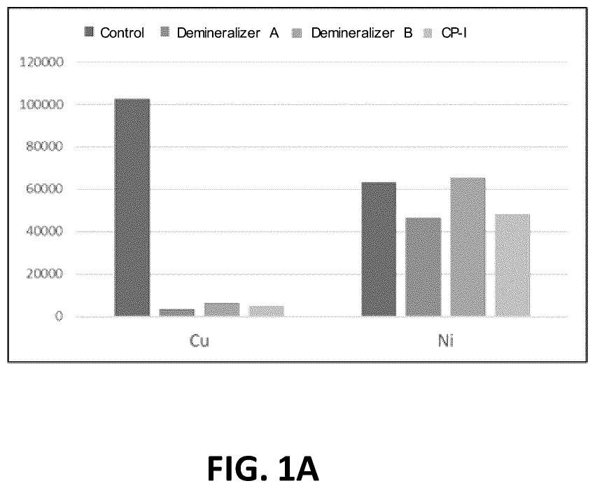

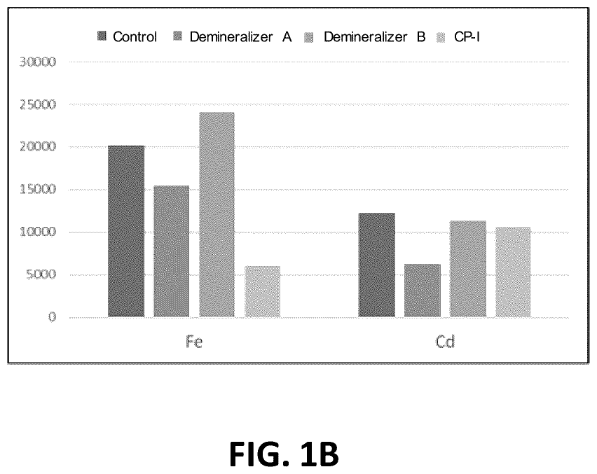

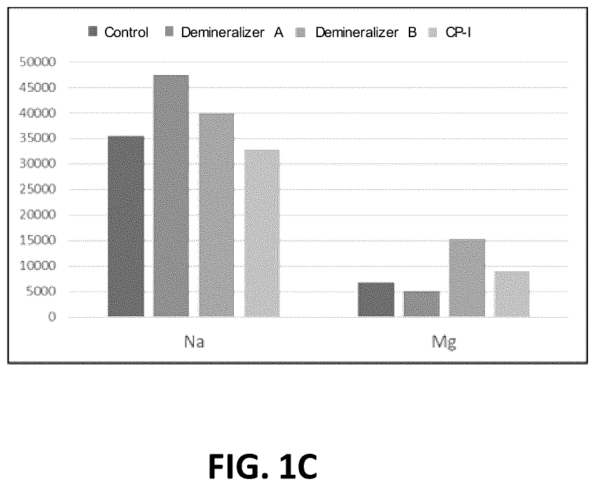

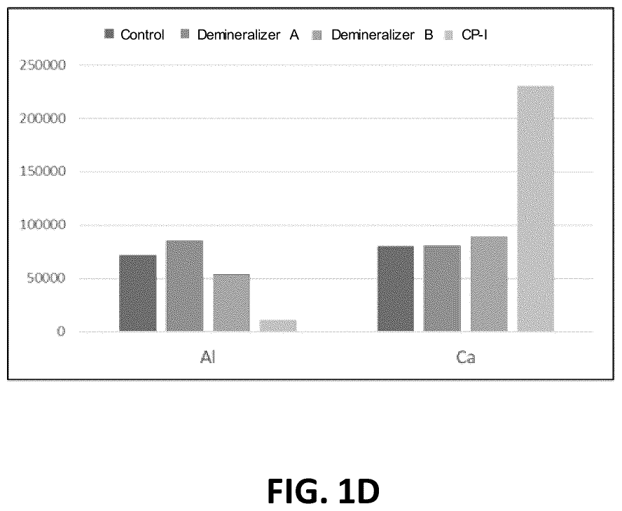

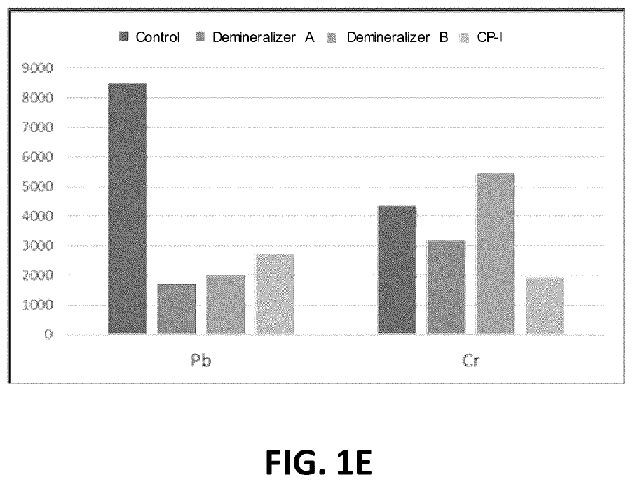

TOE-SIMS images were recorded on 3 hair fibers from bleached hair tress which were rinsed-off 10 times with “contaminated” hard water (Control), 3 hair fibers from contaminated hair tress treated with Demineralizer A, 3 hair fibers from contaminated hair tress treated with Demineralizer B, and 3 hair fibers from contaminated hair tress treated with CP-I. The average values for each element recorded on the four hair samples (FIGS. 1A-E) are summarized in the Table 2. Relative decreases (%) in the presence of metal ions are presented as compared to the control.

TABLE 2
Average values for cach element on 4 hair samples

| | | | Relative | | Relative | | Relative |
|---|---|---|---|---|---|---|---|
| Metal | | Demineralizer | decrease | Demineralizer | decrease | | decrease |
| ions | Control | A | (%) | B | (%) | CP-I | (%) |
| Fe²⁺ | 20138 | 15440 | 23% | 24071 | −20% | 6064 | 70% |
| Cu²⁺ | 102702 | 3535 | 97% | 6226 | 94% | 5121 | 95% |
| Na²⁺ | 35502 | 47419 | −34% | 39839 | −12% | 32827 | 8% |
| Al²⁺ | 71569 | 85743 | −20% | 54178 | 24% | 11121 | 84% |
| Mg²⁺ | 6792 | 5086 | 25% | 15234 | −124% | 8997 | −32% |
| Pb²⁺ | 8466 | 1707 | 80% | 1977 | 77% | 2736 | 68% |
| Cd²⁺ | 12258 | 6232 | 49% | 11369 | 7% | 10654 | 13% |
| Cr²⁺ | 4349 | 3173 | 27% | 5464 | −26% | 1889 | 57% |
| Ni²⁺ | 63386 | 46528 | 27% | 65426 | −3% | 48448 | 24% |
| Ca²⁺ | 80243 | 81057 | −1% | 89228 | −11% | 230687 | −187% |

The chelation of these elements related to the three products tested presents significant variation depending on the element analyzed. The copper and the lead were significantly decreased at the hair surface whatever the products used for the treatment. Based on these results, products Demineralizer A and CP-I were the most efficient to clean metals at the hair surface. Indeed, 7 metals decreased after treatment with CP-I and 6 metals decreased after treatment with Demineralizer A. The product Demineralizer B was still effective to clean hair surfaces from metal deposition, although less so than Demineralizer A and CP-1. The results in Table 2 illustrate that Demineralizer A was relatively more effective at removing secondary metals, including Cu²⁺, Pb²⁺, Cd²⁺, and Ni²⁺ as compared to the commercial product, CP-I. Demineralizer A was also relatively more effective at removing the primary metal, Mg²⁺, as compared to the commercial product, CP-I.

### Example 2

### Evaluation of Specific Elements on Hair Fibers by Time-of-Flight Secondary Ion Mass Spectrometry (TOF-SIMS) (II)

The presence of 11 elements on single hair fibers was evaluated before and after the application of an exemplary hair demineralizer disclosed herein.

Two samples were used to treat a 1 g hair tress, including a Demineralizer C of Table 8 (trisodium MGDA, sodium phytate, sodium gluconate, tetrasodium glutamate diacetate, tetrasodium EDTA carnosine) and CP-I. The entire packet of CP-I was mixed with 2 oz of warm water. Bleached Hair tresses were rinsed off 10 times with contaminated hard water. The hair tresses were then treated with the exemplary hair demineralizer disclosed herein at 40% of the w/w. The hair tresses were massaged for 30 seconds and left for 4 minutes before rinsing with regular water.

Samples tested include i) control hair fibers treated 10-times with contaminated hard water, ii) contaminated hair fibers treated with Demineralizer C of Table 8, and iii) contaminated hair fibers treated with CP-I.

Elements analyzed by TOF-SLMS on hair fiber surface include iron (Fe), copper (Cu), zinc (Zn), aluminum (Al), magnesium (Mg), lead (Pb), cadmium (Cd), chromium (Cr), nickel (Ni), calcium (Ca), and mercury (Hg).

TOF-SIMS makes use of the secondary ion mass spectra of atomic species or low molecular weight fragments which are formed when the sample surface is bombarded with a positively charged bismuth ion beam. Characteristic positive or negative ions, which are unique to the chemical compounds present at the point of impact of the beam, are analyzed according to the weight/charge ratio (m/z).

Three bleached hair tresses were rinsed-off 10 times with contaminated hard water. Three hair fibers were randomly selected for TOF-SIMS analysis (Control). These hair tresses were then treated with the product (Demineralizer C and CP-I) and rinsed off with regular water. Three hair fibers were randomly selected for TOF-SIMS analysis (after treatment). The samples were measured with a TOF-SIMS system (nanoTOF II, Physical Electronics) with 30 kV Bi+ ions. During analysis, low-energy electrons and Ar⁺ ions (+/−10 eV) were used to keep the sample surface charge neutral.

TOF-SIMS images were recorded on 3 single fibers from the control (Control) and the 3 treated samples (Demineralizer C of Table 8 and CP-I). The distribution of the 11 target elements (Ca, Mg, Cu, Fe, Cd, Ni, Zn, Al, Cr, and Pb) on the hair surface was achieved by calculating relative amounts of each as determined in the MS spectra.

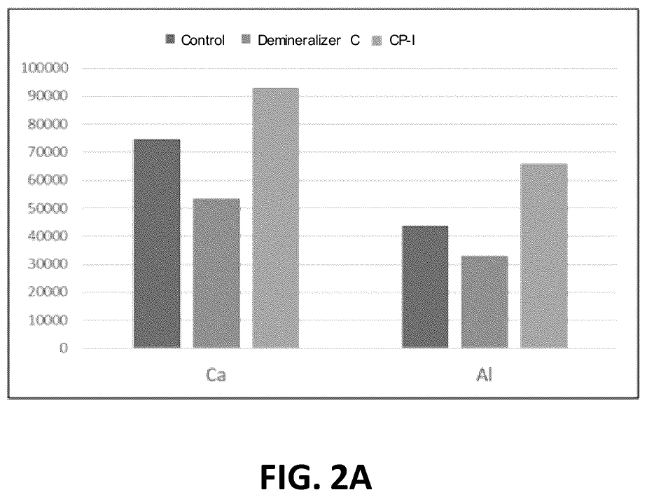

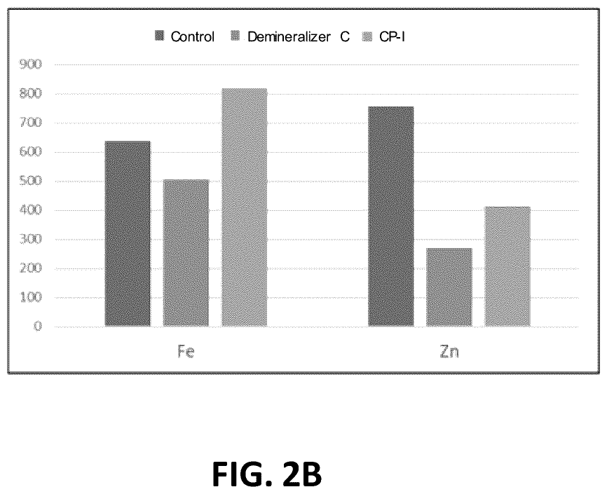

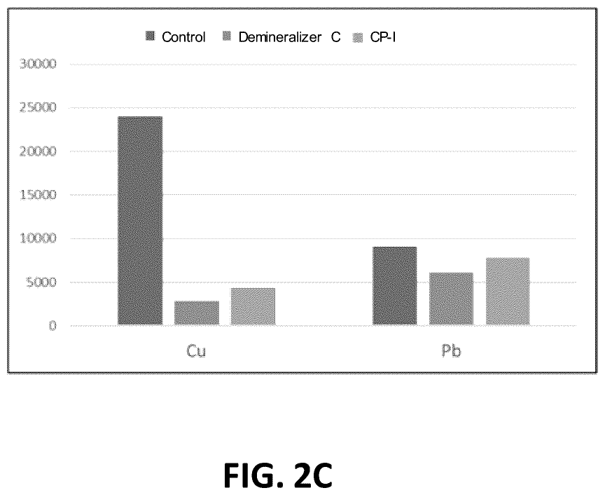

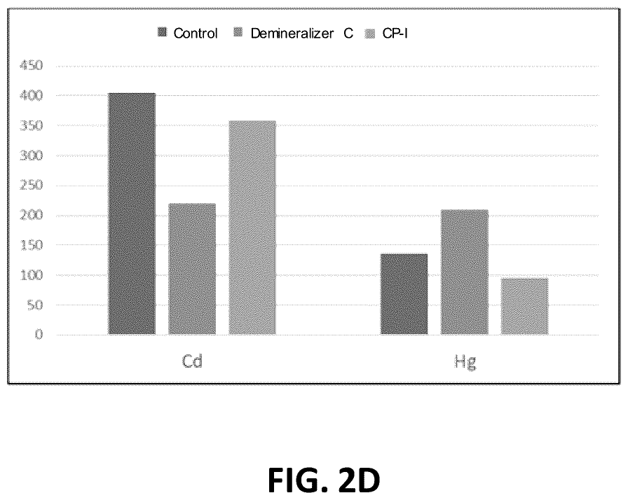

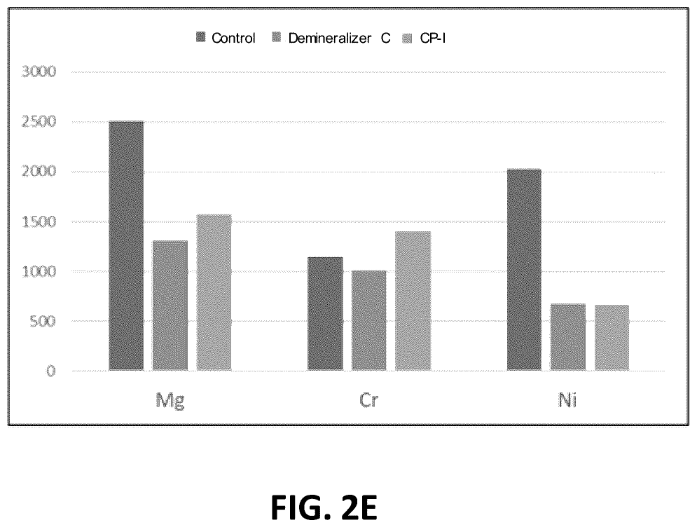

TOF-SIMS images recorded on 3 hair fibers from bleached hair tress which was rinse-off 10 times with “contaminated” hard water (Control), 3 hair fibers from contaminated hair tress treated with Demineralizer C, and 3 hair fibers from contaminated hair tress treated with CP-I. The average values for each element recorded on the three hair samples (FIGS. 2A-E) were summarized in the Table 3. Relative decrease of the presence of the metal ions is presented in Table 3 as relative to the control.

TABLE 3
Average values for each element on 3 hair samples

| | | | Relative | | Relative |
|---|---|---|---|---|---|
| | | Demineralizer C | Decrease | | Decrease |
| Metal ions | Control | of Table 8 | (%) | CP-I | (%) |
| Ca²⁺ | 74658 | 53270 | 29% | 92887 | −24% |
| Cu²⁺ | 24036 | 2859 | 88% | 4326 | 82% |
| Mg²⁺ | 2507 | 1305 | 48% | 1571 | 37% |
| Cr²⁺ | 1142 | 1013 | 11% | 1404 | −23% |
| Fe²⁺ | 636 | 504 | 21% | 818 | −29% |
| Al³⁺ | 43713 | 32830 | 25% | 65787 | −50% |
| Zn²⁺ | 756 | 268 | 65% | 412 | 46% |
| Ni²⁺ | 2023 | 678 | 66% | 669 | 67% |
| Cd²⁺ | 405 | 219 | 46% | 358 | 12% |
| Pb²⁺ | 9096 | 6091 | 33% | 7823 | 14% |
| Hg²⁺ | 136 | 209 | −54% | 95 | 30% |

The mercury was the least detected element detected on the hair fibers. The chelation of these elements related to the two products tested presents significant variation depending on the element analyzed. The copper followed by lead, nickel, manganese, and zinc were significantly decreased at the hair surface whatever the products used for the treatment. Based on these results the product Demineralizer C of Table 8 formulation is the most efficient to clean metals at the hair surface. Indeed, the concentration of 7 metals, including calcium, decreased after treatment with Demineralizer C of Table 8 formula, In this study, the product CP-I was the least effective to clean hair surface from metal deposition. As compared to CP-I, Demineralizer C of Table 8 removed more primary metals, as evidenced by the higher relative decreases of 29% and 48%, respectively of Ca²⁺ and Mg²⁺. Similarly, Demineralizer C of Table 8 also removed a larger portion of secondary metals, such as Cu²⁺, Cr²⁺, Fe²⁺, Al³⁺, Zn²⁺, Cd²⁺, and Pb²⁺, as compared to CP-I.

### Example 3

### Evaluation of Specific Elements on Hair Fibers Bar Time-of-Flidit Secondary Ion Mass Spectrometry (TOF-SIMS)

The presence of 12 elements on single hair fibers was evaluated before and after the application of a hair demineralizer disclosed herein.

Three samples were used to treat a 1 g hair tress, including a Hair Demineralizer Composition of Table 5, such as a Demineralizer C of Table 6 (10 minutes), CP-I (30 minutes), and commercial product II (CP-II) (glycine, no antioxidant) (4 minutes). The packet of CP-I included chelators (sodium gluconate, disodium EDTA), an antioxidant (ascorbic acid), and an anti-redeposition agent (xanthan gum). CP-I was a low-pH product. The entire packet of CP-I was mixed with 2 oz of warm water. Bleached hair tresses were rinsed off 10 times with contaminated hard water. The hair tresses were then treated with the exemplary hair demineralizer disclosed herein at 60% of the w/w. The hair tresses were massaged for 30 seconds and left for 4 minutes before rinsing with regular water.

Samples tested include i) control hair fibers treated 10-times with contaminated hard, water, ii) contaminated hair fibers treated with a Demineralizer C of Table 8, iii) contaminated hair fibers treated with CP-I, and iv) contaminated hair fibers treated with CP-II.

Elements analyzed by TOF-SIMS on hair fiber surfaces include iron (Fe), copper (Cu), zinc (Zn), aluminum (Al), magnesium (Mg), lead (Pb), cadmium (Cd), chromium (Cr), nickel (Ni), calcium (Ca), arsenic (As), and mercury (Hg).

TOF-SIMS makes use of the secondary ion mass spectra of atomic species or low molecular weight fragments which are formed when the sample surface is bombarded with a positively charged bismuth ion beam. Characteristic positive or negative ions, which are unique to the chemical compounds present at the point of impact of the beam, were analyzed according to the weight/'charge ratio (m/z).

3 bleached hair tresses were rinsed-off 10 times with contaminated hard water. Three hair fibers were randomly selected for TOF-SIMS analysis (Control). These hair tresses were then treated with the product (Demineralizer C of Table 8, CP-I, and CP-II) and rinsed off with regular water. Three hair fibers were randomly selected for TGF-SIMS analysis (after treatment). The samples were measured with a TOF-SIMS system (nanoTOF II, Physical Electronics) with 30 kV Bi+ ions. During analysis, low-energy electrons and Ar+ ions (+− 10 eV) were used to keep the sample surface charge neutral.

TOF-SIMS images were recorded on 3 single fibers from the control (Control) and the 3 treated samples (a Demineralizer C of Table 8, CP-I, and CP-II). The distribution of the 11 target elements (Ca, Mg, Cu, Fe, Cd, Ni, Zn, Al, Hg, Cr, and Pb) on the hair surface was achieved by calculating relative amounts of each as determined in the MS spectra.

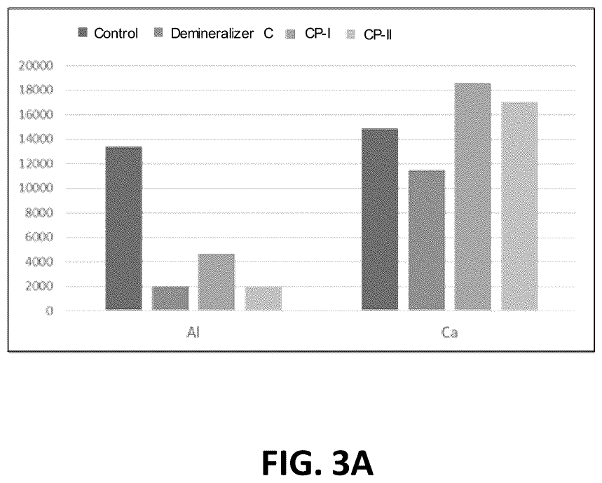

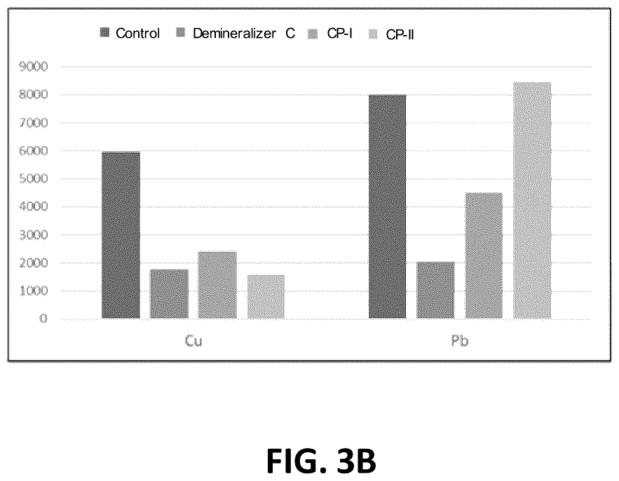

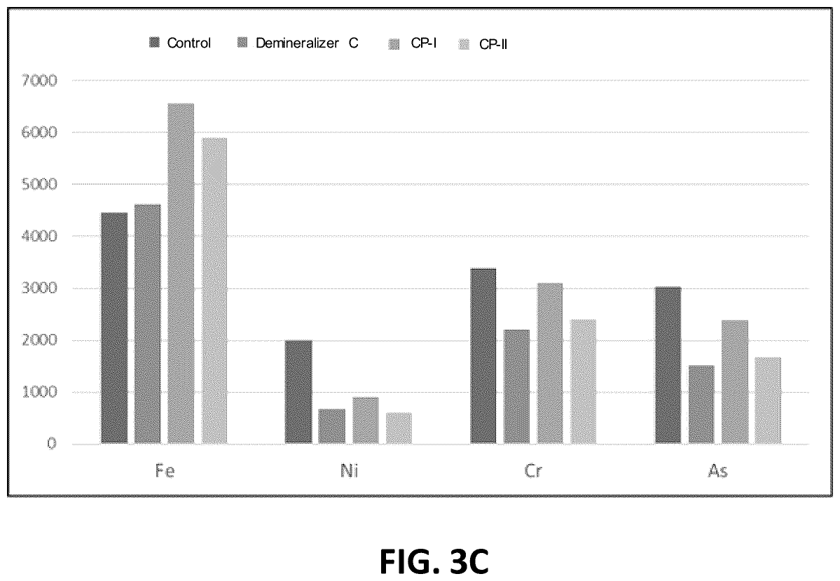

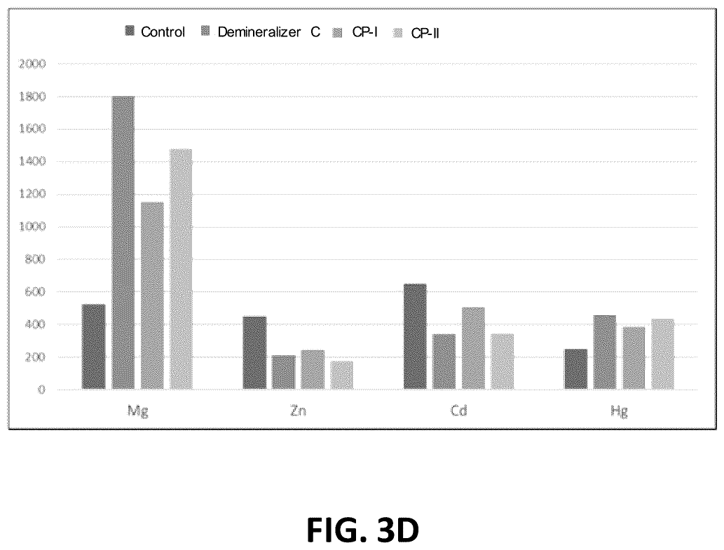

TOF-SLMS images were recorded on 3 hair fibers from bleached hair tress which were rinse-off 10 times with “contaminated” hard water (Control), 3 hair fibers from contaminated hair tress treated with a Demineralizer C of Table 8, 3 hair fibers from contaminated hair tress treated with CP-I, and 3 hair fibers from contaminated hair tress treated with CP-II. The average values for each element recorded on the four hair samples (FIGS. 3A-D) were summarized in the Table 4. CP-I was as described above, and CP-II had one chelator (glycine),

TABLE 4
Average values for each element on 3 hair samples

| | | | Relative | | Relative | | Relative |
|---|---|---|---|---|---|---|---|
| Metal | | Demineralizer C | Decrease | | Decrease | | Decrease |
| ions | Control | of Table 8 | % | CP-I | % | CP-II | % |
| Cu²⁺ | 5960 | 1767 | 70% | 2407 | 60% | 1579 | 74% |
| Ca²⁺ | 14888 | 11462 | 23% | 18548 | −25% | 17034 | −14% |
| Mg²⁺ | 525 | 1800 | −243% | 1150 | −119% | 1477 | −181% |
| Al³⁺ | 13386 | 2012 | 85% | 4698 | 65% | 1929 | 86% |
| Fe²⁺ | 4453 | 4611 | −4% | 6557 | −47% | 5889 | −32% |
| Cr²⁺ | 3384 | 2212 | 35% | 3098 | 8% | 2402 | 29% |
| Zn²⁺ | 450 | 207 | 54% | 244 | 46% | 177 | 61% |
| Ni²⁺ | 1986 | 668 | 66% | 904 | 54% | 596 | 70% |
| Cd²⁺ | 649 | 341 | 47% | 508 | 22% | 344 | 47% |
| Pb²⁺ | 8007 | 2028 | 75% | 4507 | 44% | 8460 | −6% |
| Hg²⁺ | 248 | 460 | −85% | 383 | −54% | 434 | −75% |
| As²⁺ | 3029 | 1505 | 50% | 2377 | 22% | 1679 | 45% |

While the chemical ingredients to prepare the custom hard water were changed, hoping to improve visualization of some of the harder-to-image metals, mercury (Hg²⁺) was still clearly the lowest element detected on the hair fibers. The chelation of these metals related to the three products tested presents significant variation depending on the metals analyzed. The copper and the aluminum followed by the nickel, and the zinc were significantly decreased at the hair surface whatever the products used for the treatment Based on these results the product Demineralizer C formulation was the most efficient to clean metals at the hair surface. Indeed, the concentration of 8 metals, including calcium, decreased after treatment with Demineralizer of Table 8. The relative decrease (%) of the different metal ions in the hair is presented above in Table 4 as compared to the control. In this study CP-I is the least effective to clean hair surfaces from metal deposition.

### Example 4

### Preparation of Hair Demineralizer

Hair demineralizer compositions are provided in Tables 5-8. The exemplary hair demineralizer compositions are made by formulation and mixing techniques or by mixing together solvent (water), chelators, surfactant, anti-redeposition polymer or rheology modifier, antioxidant, pH modifier, and preservatives at an elevated temperature. The hair demineralizer compositions were as described in Table 5. The ingredients are mixed thoroughly at elevated temperature and then cooled to ambient temperature. In some cases, the ingredients are mixed completely at room temperature. Additional ingredients may be added to the cooled product. The amount stated reflects the weight percent of the active material, unless otherwise specified. The pH for Demineralizer D, Demineralizer E, and Demineralizer C were each about 7 and about 8.

TABLE 5
Hair Demineralizer Compositions

| | Percent formula (% w/w) | | | | |
|---|---|---|---|---|---|
| Ingredient & | Composition | Composition | Composition | Composition | Composition |
| Function | #1 | #2 | #3 | #4 | #5 |
| Solvent | QSP | QSP | QSP | QSP | QSP |
| Surfactant/ | 0.01-2 | 0.01-2 | 0.1-0.5 | 0 | 0 |
| Solubilizer | | | | | |
| Antioxidant | 1-2 | 0.1-3 | 1-3 | 0.005-5 | 0.1-1 |
| Demineralizer | 0.025-25 | 0.1-2 | 0.01-5 | 0.015-25 | 0.025-5 |
| Preservative | 0.005-0.5 | 0.1-9 | 0.5-5 | 0 | 0,005-0.1 |
| Anti- | 0.3-0.5 | 0.1-1 | 0 | 0 | 0.005-1 |
| Redeposition | | | | | |
| Agent | | | | | |
| pH Modifier | 0.2 | 0.3 | 0.5 | 0 | 1.5-2 |
| pH | 6-9 | 6-8 | 7-9 | 6-9 | 6-9 |

TABLE 6
Hair Demineralizer D compositions

| | | Demineralizer | Demineralizer | Demineralizer | Demineralizer |
|---|---|---|---|---|---|
| | | D-1 | D-2 | D-3 | D-4 |
| | | Percent | Percent | Percent | Percent |
| | | formula | formula | formula | formula |
| Ingredient | Function | (% w/w) | (% w/w) | (% w/w) | (% w/w) |
| Water | Solvent | 93.292 | 88.802 | 90.156 | 93.292 |
| Polysorbate | Surfactant/ | 0 | 2.3 | 0.8 | 0 |
| 20 | Solubilizer | | | | |
| Carnosine | Antioxidant | 1 | 2.2 | 0.75 | 1 |
| Trisodium | Chelator | 1 | 0.8 | 1.5 | 1 |
| Dicarboxymethyl | | | | | |
| Alaninate | | | | | |
| Sodium | Chelator | 0 | 0.8 | 0.9 | 0 |
| Phytate | | | | | |
| Sodium | Chelator | 0 | 0 | 0.66 | 0 |
| Gluconate | | | | | |
| Tetrasodium | Chelator | 1 | 1.15 | 1.1 | 1 |
| Glutamate | | | | | |
| Diacetate | | | | | |
| Tetrasodium | Chelator | | 1.02 | 0.61 | |
| EDTA | | | | | |
| Sodium | Anti- | 0 | 0.87 | 0.6 | 0 |
| Carboxymet | Deposition | | | | |
| hyl Inulin | Agent | | | | |
| Sapindus | Cleanser | 0.5 | 0.5 | 0.4 | 0.5 |
| mukorossi | | | | | |
| Peel Extract | | | | | |
| Lactic Acid | pH Modifier | 0.15 | 0.28 | 0.15 | 0.15 |
| Gluconolactone | Preservative | 1 | 0.4 | 1.3 | 1 |
| Sodium | Preservative | 0.2 | 0.015 | 0.22 | 0.2 |
| Benzoate | | | | | |
| Calcium | Preservative | 0.01 | 0.015 | 0.006 | 0.01 |
| Gluconate | | | | | |
| Fragrance | Perfume | 0.758 | 0 | 0 | 0 |
| (Parfum) | | | | | |
| Limonene | Perfume | 0.027 | 0 | 0 | 0 |
| Hydroxycitronellol | Perfume | 0.016 | 0 | 0 | 0 |
| Benzyl | Perfume | 0.015 | 0 | 0 | 0 |
| Salicylate | | | | | |
| Hexyl | Perfume | 0.032 | 0 | 0 | 0 |
| Cinamal | | | | | |

TABLE 7
Hair Demineralizer E compositions

| | | Demineralizer | Demineralizer | Demineralizer | Demineralizer |
|---|---|---|---|---|---|
| | | E-1 | E-2 | E-3 | E-4 |
| | | Percent | Percent | Percent | Percent |
| | | formula | formula | formula | formula |
| Ingredient | Function | (% w/w) | (% w/w) | (% w/w) | (% w/w) |
| Water | Solvent | 90.551 | 86.992 | 86.907 | 90.239 |
| Polysorbate 20 | Surfactant/ | 2.400 | 4.5 | 3.7 | 2.2 |
| | Solubilizer | | | | |
| Carnosine | Antioxidant | 1.000 | 1 | 1 | 1 |
| Trisodium | Chelator | 1.100 | 0 | 0.75 | 0.27 |
| Dicarboxymethyl | | | | | |
| Alaninate | | | | | |
| Sodium Phytate | Chelator | 0.550 | 2.75 | 0.55 | 1.55 |
| Sodium | Chelator | 0.700 | 0 | 1.5 | 0 |
| Gluconate | | | | | |
| Tetrasodium | Chelator | 0.594 | 0 | 0.53 | 0.65 |
| Glutamate | | | | | |
| Diacetate | | | | | |
| Tetrasodium | Chelator | 0.413 | 0.55 | 0 | 0.887 |
| EDTA | | | | | |
| Gluconolactone | Preservative | 0.500 | 1 | 0 | 0.75 |
| Sodium | Anti- | 0.600 | 0 | 0.6 | 0 |
| Carboxymethyl | Deposition | | | | |
| Inulin | Agent | | | | |
| Sapindus | Cleanser | 0.400 | 0.4 | 0.4 | 0.4 |
| mukorossi Peel | | | | | |
| Extract | | | | | |
| Lactic Acid | pH | 0.178 | 1.5 | 1.25 | 1 |
| | Modifier | | | | |
| Sodium | Preservative | 0.188 | 0.5 | 2 | 0.25 |
| Benzoate | | | | | |
| Calcium | Preservative | 0.028 | 0.01 | 0.015 | 0.006 |
| Gluconate | | | | | |
| Fragrance | Perfume | 0.658 | 0 | 0 | 0 |
| (Parfum) | | | | | |
| Limonene | Perfume | 0.087 | 0 | 0 | 0 |
| Hydroxycitronellol | Perfume | 0.006 | 0 | 0 | 0 |
| Benzyl | Perfume | 0.015 | 0 | 0 | 0 |
| Salicylate | | | | | |
| Hexyl Cinamal | Perfume | 0.032 | 0 | 0 | 0 |

TABLE 8
Hair Demineralizer C compositions

| | | Demineralizer | | Demineralizer | Demineralizer | Demineralizer |
|---|---|---|---|---|---|---|
| | | C-1 | | C-2 | C-3 | C-4 |
| | | Percent | | Percent | Percent | Percent |
| | | formula | | formula | formula | formula |
| Ingredient | Function | (% w/w) | Ranges | (% w/w) | (% w/w) | (% w/w) |
| Water | Solvent | 90.54900 | QSP | 89.93 | 84.315 | 91.87 |
| Polysorbate | Surfactant/ | 2.00000 | 0-5 | 2.5 | 5 | 1 |
| 20 | Solubilizer | | | | | |
| Carnosine | Antioxidant | 1.00000 | 0.005-5 | 1 | 5 | 2 |
| Trisodium | Chelator | 1.00000 | 0.005-5 | 0.5 | 1.5 | 0.25 |
| Dicarboxymethyl | | | | | | |
| Alaninate | | | | | | |
| Sodium | Chelator | 0.95000 | 0.005-5 | 1.5 | 0.005 | 0.1 |
| Phytate | | | | | | |
| Sodium | Chelator | 0.80000 | 0.005-5 | 0 | 0 | 0.66 |
| Gluconate | | | | | | |
| Tetrasodium | Chelator | 0.59400 | 0.005-5 | 0.66 | 0.66 | 0.594 |
| Glutamate | | | | | | |
| Diacetate | | | | | | |
| Tetrasodium | Chelator | 0.51300 | 0.005-5 | 1 | 1.02 | 0.45 |
| EDTA | | | | | | |
| Gluconolactone | Preservative | 0.50000 | 0.1-2 | 0 | 0.87 | 0.25 |
| Sodium | Anti- | 0.50000 | 0.005-5 | 0.5 | 0 | 0.1 |
| Carboxymethyl | Deposition | | | | | |
| Inulin | Agent | | | | | |
| Sapindus | Cleanser | 0.40000 | 0-5 | 0.4 | 0.4 | 0.4 |
| mukorossi | | | | | | |
| Peel Extract | | | | | | |
| Lactic Acid | pH Modifier | 0.19800 | 0-2 | 1 | 0.4 | 1.3 |
| Sodium | Preservative | 0.18800 | 0-5 | 0.2 | 0.015 | 0.22 |
| Benzoate | | | | | | |
| Calcium | Preservative | 0.00800 | 0-2 | 0.01 | 0.015 | 0.006 |
| Gluconate | | | | | | |
| Fragrance | Perfume | 0.65874 | 0-5 | 0 | 0 | 0 |
| (Parfum) | | | | | | |
| Limonene | Perfume | 0.07746 | 0-5 | 0 | 0 | 0 |
| Hydroxycitronellol | Perfume | 0.03670 | 0-5 | 0 | 0 | 0 |
| Benzyl | Perfume | 0.01511 | 0-5 | 0 | 0 | 0 |
| Salicylate | | | | | | |
| Hexyl | Perfume | 0.01200 | 0-5 | 0 | 0 | 0 |
| Cinamal | | | | | | |

### Example 5

### Swimmer's Hair Color Prevention/removal

Ten European medium brown tresses (3.0 g, 8″ length, 1″ wide), 9% paste bleached, were used per treatment group.

Bleaching treatment protocol using Clairol Professional BW2 lightener and Salon Care clear 30 vol developer as follows: First, powder bleach with developer was mixed in a 13:22 weight ratio to bleach 6 tresses per batch so it would yield 15 g per tress. The mixture was applied evenly using a brush on both sides of the tress, massaged using fingers into the hair. The foil was closed and tresses were placed into an oven at 40+2° C. for 30 min. The bleach was washed thoroughly washed out of tresses with manual manipulation for 2 minutes under a tap water at 40° C. with a controlled flow rate of 1.0 GPM. Hair was blow-dried using medium heat, high-velocity settings. After the tresses were dried, one more bleaching cycle was performed. The bleach was thoroughly washed out of tresses with manual manipulation for 2 minutes under a tap water set at 40° C. with a controlled flow rate of 1.0 GPM. 0.3 g of 15% SLES was applied per 1 tress of hair. The hair was massaged for 30 s and rinsed for 30 s. All tresses were dried overnight at 60+2% and 20+2° C.

A total of three test groups (3 cells) have been tested: Test group 1 (Cell 1) was a control with pool treatment alone and no washing. Test group 2 (Cell 2) used a Demineralizer C of Table 8 only once after 5 times treatment cycle and Test group 3 (Cell 3) used a representative Demineralizer C of Table 8 disclosed herein with detox shampoo only once after 5 times treatment cycle.

Each treatment was performed using an Intelllifaucet set at approximately 40° C. and 1.0 GPM flow rate. The extreme imitation pool water consisted of deionized (DI) water, 4 ppm chlorine and 0.5 ppm copper (copper sulfate for pools manufacturer (AquaVet) recommended to use 1 ppm copper for pools, but this was too severe coloring). All test groups were soaked in simulated pool water for one hour and then processed as follows: Test group 1 (Cell 1) allowed hair to air dry before beginning next cycle and color readings were taken on dry hair after 5 treatment cycles. Test group 2 (Cell 2) allowed hair to air dry before beginning next cycle. After 5^{th} cycle only, on dry hair, 60% w/w chelator was applied to hair tress. The hair was massaged for 30 seconds, left on for 4 minutes, rinsed for 30 seconds, and allowed to air dry before color readings. Color readings were taken on dry hair after 5 pool treatment cycles followed by single treatment with chelator. Test group 3 (Cell 3) allowed hair to air dry before beginning next cycle. After 5^{th} cycle only, on dry hair, 60% w/w chelator was applied to hair tress. The hair was massaged for 30 seconds, left on for 4 minutes, and rinsed for 30 seconds. On damp hair, 10% w/w shampoo was applied to hair tress. The hair was massaged for 30 seconds, rinsed for 30 seconds, and allowed to air dry before color readings. Color readings were taken on dry hair after 5 treatment cycles followed by single treatment with chelator washed with detox. For each treatment set, tresses were allowed to dry overnight at 22±2° C., 60±5% RH (relative humidity) before the next cycle.

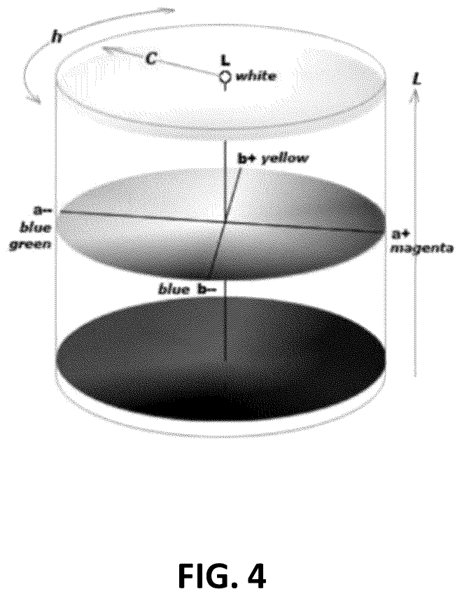

All the bleached hair tresses were labeled to allow for identification, Baseline L, a, b measurements were taken using a Hunter Lab IJltraScan VIS colorimeter to characterize the initial color of hair. Technical hair color was often quantified using the CIELAB L, a, b system (parameters further explained in the appendix). That color is represented in a 3-dimensional matrix where “L” refers to the lightness on a scale of 0 to 100, “a” denotes the red-green color range (positive value denotes higher red) and “b” represents the yellow-blue color range (positive value denotes higher yellow). This is demonstrated in FIG. 4. Generally, changes in the color of the tress were evaluated by calculating the difference in these parameters relative to a reference state (i.e. ΔL*, Δa*, and Δb*). In addition, it was common to report an overall color change, ΔE. That is,

ΔE=√[ΔL*²+Δa*²+Δb*²]

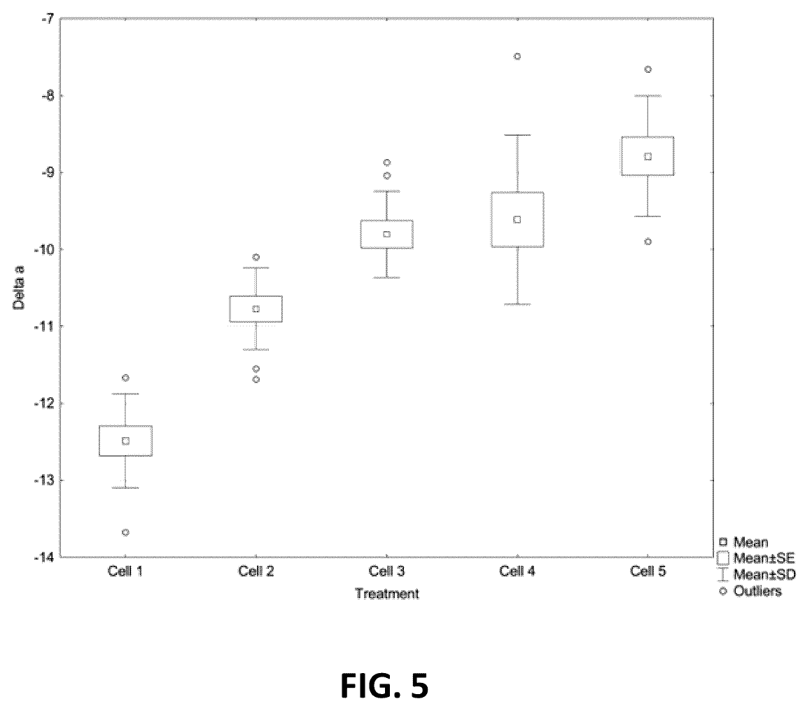

However, for this study, delta a parameter was used to evaluate the greenness of the hair. Ten (10) color measurements were performed each tress. Ten (10) tresses per treatment provided appropriate statistical rigor. Box and whisker plot was generated using Statistica™, while JMP™ analytical software was used to calculate the statistics (student's t-test at 95% confidence level). Data are given in the Appendix. Results from testing are shown in FIG. 5 and Table 9A.

TABLE 9A
Results of color changes

| Treatment | N | Mean | Std. Dev. | Std. Err. Mean |
|---|---|---|---|---|
| Cell 3 | 10 | −8.8 | 0.8 | 0.2 |
| Cell 2 | 10 | −9.6 | 1.1 | 0.3 |
| Cell 1 | 10 | −12.5 | 0.6 | 0.2 |

The followings reduce the greenness of simulated pool water-treated hair when compared with untreated: usage of a representative chelator disclosed herein with detox shampoo only once after 5 times treatment cycle and usage of a representative chelator disclosed herein only once after 5 times treatment cycle.

CIELAB L*a*b* parameters: “L” scale measures the lightness or grayness component of the colored sample, with L=0 for black and L=100 for white. ΔL values (difference between after and before treatment) indicate either a lighter (positive values) or a darker (negative values) sample resulting from any treatment protocol. “a” scale measures the chromaticity differences in the red-green components of the color observed. Positive changes in a are correlated with increases in the red component. “b” scale pertains to chromaticity differences in the yellow-blue components, positive changes in it being an indication of increased yellowness. Using these L, a, b values, it is possible to calculate other parameters. Changes in C (denoted by ΔC) represent changes in chroma. ΔE is a measure of the total color difference between the sample (after) and sample (before). ΔH indicates changes in hue,

C=(a²+b²)^{0:5}

ΔC=(C_{after}-C_{before})

ΔL=L_{after}-L_{before}

ΔE=[(ΔL)²+(Δa)²+Δb)2]^{0.5}

ΔH=[(ΔE)²−(ΔL)²−(ΔC)²]^{0.5}

ΔL is a good monitor of fading, since it is a brightness parameter. It is seen that ΔE, the parameter representing color changes, is a very good monitor of the fading phenomenon, since changes in it are, in fact, a “composite” of changes in brightness and color. It incorporates more than one type of change in the fading phenomenon. Hence, changes in it are a good indicator of color fastness under any treatment protocol studied.

### Example 6

### Color Uptake Testing

Twenty European medium brown tresses (3.0 g, 8″ length, 1″ wide), soaked in contaminated hard water, and twenty European medium brown tresses (3.0 g, 8″ length, 1″ wide), bleached and soaked in contaminated hard water, were used per treatment group. The contaminated hard water contained both transition metals and calcium and magnesium salts.

Bleaching treatment protocol: Mix 1 part Clairol Professional BW2 Hair Powder Lightener to 2 parts Salon Care 40 volume Creme Developer. Add 10 grams of mixture to each hair tress using a brush (5 g on each side) and massage thoroughly into tress. Position tresses on a hot plate with temperature maintained at 27° C. Allow the bleach to process on the hair for 40 minutes at 27° C. Turn tresses over after 20 min (halfway) and massage to allow for even coloring. Rinse out under an Intellifaucet for 2 minutes (or until water runs clear) at 40° C. and 1.0 GPM flow rate. Allow tresses to rest for at least 12 hours.

Coloring treatment protocol: The hair tresses were color treated according to the procedure with a commercial coloring agent. Combine color components according to the manufacturer's instructions and mix thoroughly. Add 10 grams of mixture to each hair tress using a brush (5 g on each side) and massage thoroughly into tress. Position tresses on a hot plate with temperature maintained at 27° C. Allow the color to process on the hair for 20 minutes at 27° C. Turn tresses over after 10 min (halfway) and massage to allow for even coloring. Rinse out under an Intellifaucet for 2 minutes (or until water runs clear) at 40° C. and 1.0 GPM flow rate. Allow tresses to rest for at least 12 hours prior to taking post color readings.

A total of four test groups (4 cells) were tested: Test group 1 (Cell 1) was treated by contaminated hard water and bleach (on virgin hair); Test group 2 (Cell 2) was treated by contaminated hard water and Demineralizer C of Table 8 and bleach (on virgin hair); Test group 3 (Cell 3) was treated by contaminated hard water and color (on bleached hair); and Test group 4 (Cell 4) was treated by contaminated hard water and Demineralizer C of Table 8 Demineralizer C of Table 8 and color (on bleached hair).

Each treatment was performed using an Intellifaucet set at approximately 40° C. and 1.0 GPM flow rate. Photos of all tresses have been taken three times including before contaminated hard water treatment, after contaminated hard water treatment, and after bleach/color application. More detailed procedures for test groups are as follows.

Cells 1 and 2 were tested for color (L*A*B) change with treatment. The substrate for each cell was 3 g, 8″ long, 1″ wide European medium brown tresses, soaked in contaminated hard water, 10 tresses per cell were used. Measurement time-points occurred after contaminated hard water soaking and after treatment. Cell 1 was treated by contaminated hard water+water+bleach. Cell 2 was treated by contaminated hard water+Demineralizer C of Table 8+bleach.

Contaminated hard water treatment involved soaking for 5 minutes followed by blow drying at a low temperature and repeating 10 times. The hair was allowed to dry and equilibrate at 60% RH. For applying chelator (or water for control cells), on dry hair, 60% w/w Demineralizer C of Table 8 was applied to hair tress. The hair was messaged for 30 seconds, left on for 4 minutes and do not rinse, and allowed to air dry before bleaching or coloring. Bleach application was performed as follows: Mix 1 part Clairol Professional BW2 Hair Powder Lightener to 2 parts Salon Care 40 volume Crème Developer. Add 10 grams of mixture to each hair tress using a brush (5 g on each side) and massage thoroughly into tress. Position tresses on a hot plate with temperature maintained at 27° C. Allow the bleach to process on the hair for 40 minutes at 27° C. Turn tresses over after 10 min (halfway) and massage to allow for even coloring. Rinse out under an Intellifaucet for 2 minutes (or until water runs clear) at 40° C. and 1.0 GPM flow rate. And allow tresses to rest for at least 12 hours prior to taking post bleach readings.

Cells 3 and 4 were tested for color (L*A*B) change with treatment. The substrate for each cell was 3 g, 8″ long, 1″ wide European medium brown tresses, bleached and soaked in contaminated hard water. For each cell, 10 tresses were tested. Measurement time-points occurred after contaminated hard water soaking and after treatment, Cell 3 was treated by Contaminated hard Water+Water+Color. Cell 4 was treated by Contaminated hard Water+Demineralizer C of Table 8 Color. Contaminated hard water treatment involved soaking for 5 minutes followed by blow drying at a low temperature and repeating 10 times. The hair was allowed to dry and equilibrate at 60% RH. Bleach application was performed as follows: Mix 1 part Clairol Professional BW2 Hair Powder Lightener to 2 parts Salon Care 40 volume Creme Developer, Add 10 grams of mixture to each hair tress using a brush (5 g on each side) and massage thoroughly into tress. Position tresses on a hot plate with temperature maintained at 27° C. Allow the bleach to process on the hair for 40 minutes at 27° C. Turn tresses over after 10 min (halfway) and massage to allow for even coloring. Rinse out under an Intellifaucet for 2 minutes (or until water runs clear) at 40° C. and 1.0 GPM flow rate. Allow tresses to rest for at least 12 hours. Chelator application was performed as follows (or water for control cells): on dry hair, apply 60% w/w Demineralizer C of Table 8 to hair tress. Massage for 30 seconds. Leave on for 4 minutes, do not rinse. And allow hair to air dry before bleaching or coloring. Color application was performed as follows: The hair tresses are color treated according to the procedure below with a commercial product. Combine color components according to the manufacturer's instructions and mix thoroughly. Add 10 grams of mixture to each hair tress using a brush (5 g on each side) and massage thoroughly into tress. Position tresses on a hot plate with temperature maintained at 27° C. Allow the color to process on the hair for 20 minutes at 27° C. Turn tresses over after 10 min (halfway) and massage to allow for even coloring. Rinse out under an Intellifaucet for 2 minutes (or until water runs clear) at 40° C. and 1.0 GPM flow rate. Allow tresses to rest for at least 12 hours prior to taking post color readings. Tresses are allowed to equilibrate overnight at 22±2° C., 60±5% RH before taking time-point measurements after hard water soaking and after colorlblea.ch treatment.

Color Fade Testing: AR the dyed hair tresses were labeled to allow for identification. Baseline L, a, b measurements were taken using a Hunter Lab UltraScan VIS colorimeter to characterize the initial color of hair. Technical hair color is often quantified using the CIELAB L, a, b system (parameters further explained in the appendix). That color is represented in a 3-dimensional matrix where “L” refers to the lightness on a scale of 0 to 100, “a” denotes the red-green color range (positive value denotes higher red) and “b” represents the yellow-blue color range (positive value denotes higher yellow). This is demonstrated in the schematic of FIG. 4. Generally, changes in the color of the tress were evaluated by calculating the difference in these parameters relative to a reference state (i.e. ΔL*, Δa*, and Δb*), In addition, it was common to report an overall color change, ΔE. That is,

ΔE=√[ΔL*²+Δa*²+Δb*²]

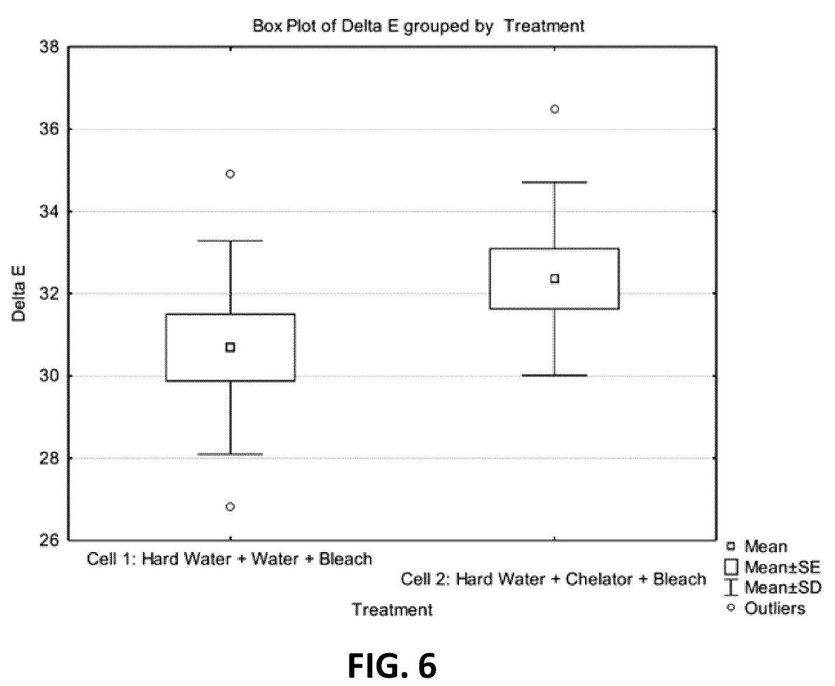

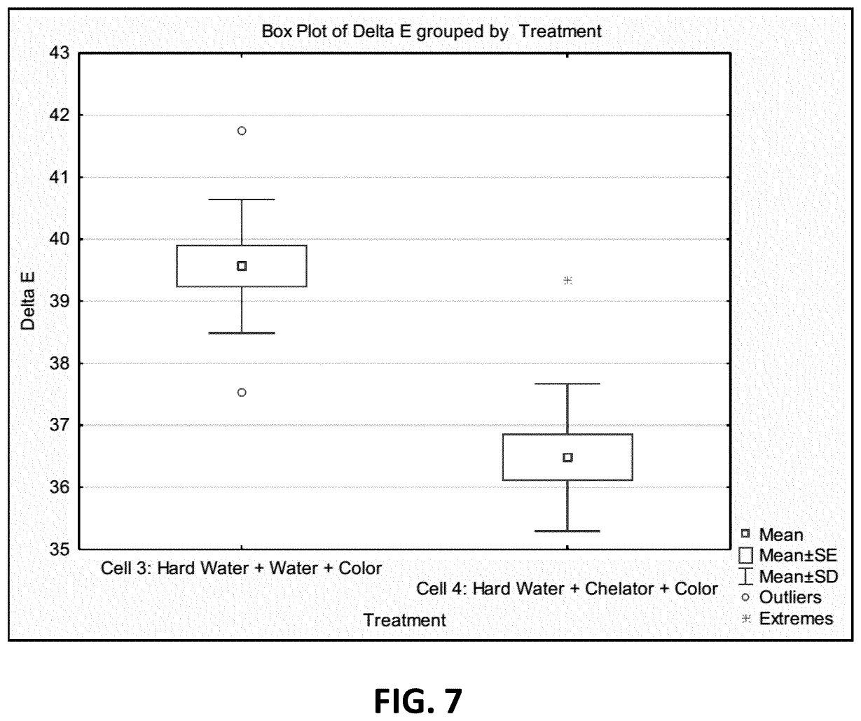

Ten (10) color measurements were performed each tress, Ten (10) tresses per treatment provided appropriate statistical rigor. Box and whisker plot was generated using Statistica™, while JMP™ analytical software was used to calculate the statistics (student's t-test at 95% confidence level). Data are given in the Appendix. Results from testing are shown in FIG. 6, FIG. 7, and Table 9B.

TABLE 9B
Results of color changes

| Treatment | N | Mean | Std. Dev. | Std. Err. Mean | Differences |
|---|---|---|---|---|---|
| Cell 2 | 10 | 32.36 | 2.35 | 0.74 | A |
| Cell 1 | 10 | 30.69 | 2.59 | 0.82 | A |
| Cell 3 | 10 | 39.56 | 1.08 | 0.34 | A |
| Cell 4 | 10 | 36.48 | 1.19 | 0.37 | B |

After soaking tresses in contaminated hard water and bleaching, there was no statistical difference in overall color change in Cells 1 & 2, ΔE, between tresses treated with the DI water vs tresses treated with a chelator. This demonstrates that the Demineralizer C of Table 8 did not affect the level of lift, volume, or processing time for a bleach/lightening service.

After soaking tresses in contaminated hard water and coloring, there is a statistical difference in overall color change, NE, when comparing Cells 3 & 4, that were treated with DI water versus tresses treated with a Demineralizer C of Table 8, The application of hair dye on the untreated hair was noticeably darker than the application of hair dye on hair treated with chelator, This demonstrates that use of the Demineralizer C of Table 8 facilitates optimal color deposit for truer, more vibrant color.

### Example 7

### Metal Chelation of Formulations by Qualitative Water Test Strip Evaluation

Test Solutions, Test Strips, and Evaluation—50 g of each solution was pre-dispensed into medium sized weigh boats for test strip evaluation. For positive and negative controls, pure 100% solutions were measured, contaminated hard water (as detailed in above section) and ASTM Type lI deionized water, respectively. For test groups, a solution of contaminated hard water and chelator formula were premixed prior to test strip evaluation, as follows: 30 g of the contaminated hard water and 20 g of the chelator formula were dispensed into weigh boats and mixed by hand for 10 seconds. This was determined to be the most effective method to qualify chelator performance via the test strip visual,

Water test strips with color indicator panels illustrating water hardness as well as presence of certain trace metals were obtained from Amazon. Per directions on the water testing kits, each strip was immersed in the solution to be tested for approximately 2. seconds then removed. Color comparison evaluations were performed after 15 seconds out of solution, noting colors are no longer representative once dried. Standard representative color calibrations were depicted on the side of the test strip container to approximate levels of each depicted compound. Color comparisons were made qualitatively by eye and translated into data reported in Table 10A. Data reports performance of the compositions and formulas as reduction in metals detected vs positive control—a greater number of asterisks indicates a larger change in metals detected (a more significant decrease) vs contaminated hard water (CHW) control, as described in Table 10B.

Results of the test strip chelation experiment are shown in Table 10A, and the key for interpreting the results in Table 10A is presented in Table 10B. The study has shown that differently altered compositions of Table 10A have various degrees of metal sequestration as illustrated using color indicator water test strips.

TABLE 10A
Qualitative Water Test Strip Results of Formulations

| | Tested | Percent in Formula (% w/w) | | | | | | | | | | | |
|---|---|---|---|---|---|---|---|---|---|---|---|---|---|
| | Formula | | | | | | | | | | Hair | | |
| | Attributes | | | | | | | | | | Deminer- | | |
| | & Results | | | | | | | | | | alizer | Control - | |
| | Formula | | | | | | | | | | Compo- | DI | Control - |
| Version | Number | 1 | 2 | 3 | 4 | 5 | 6 | 7 | 8 | 9 | sition | Water | CHW |
| Ingre- | Water | QS | QS | QS | QS | QS | QS | QS | QS | QS | Referenced | 100% | — |
| dients | Carnosine | 1.0 | 1.0 | 1.0 | 1.0 | 1.0 | 1.0 | 1.0 | 1.0 | 1.0 | in Table 8 | — | |
| | Trisodium | 1.0 | 1.0 | 1.0 | 1.0 | 0 | 0 | 1.0 | 1.0 | 1.0 | | | |
| | MGDA | | | | | | | | | | | | |
| | Sodium | 0.5 | 0.5 | 0.5 | 0.5 | 0 | 0 | 0 | 0 | 0 | | | |
| | phytate | | | | | | | | | | | | |
| | Sodium | 0.5 | 0.5 | 0.5 | 0.5 | 0 | 0.5 | 0.5 | 0.5 | 0 | | | |
| | gluconate | | | | | | | | | | | | |
| | Tetra- | 0.4 | 0.4 | 0.4 | 0.4 | 0 | 0 | 0 | 0.4 | 0.4 | | | |
| | sodium | | | | | | | | | | | | |
| | GLDA | | | | | | | | | | | | |
| | Tetra- | 0.4 | 0.4 | 0.4 | 0.4 | 0.4 | 0.4 | 0.4 | 0.4 | 0.4 | | | |
| | sodium | | | | | | | | | | | | |
| | EDTA | | | | | | | | | | | | |
| | Sodium | 2.0 | 0 | 2.0 | 0 | 0 | 0 | 0 | 0 | 2.0 | | | |
| | carboxy- | | | | | | | | | | | | |
| | methyl | | | | | | | | | | | | |
| | inulin | | | | | | | | | | | | |
| Formula | pH | 4.0-4.5 | 4.0-4.5 | 7.5-8.0 | 7.5-8.0 | 7.5-8.0 | 7.5-8.0 | 7.5-8.0 | 7.5-8.0 | 7.5-8.0 | 7.5-8.0 | — | — |
| pH | | | | | | | | | | | | | |
| CHW | See | Y | Y | Y | Y | Y | Y | Y | Y | Y | Y | N | Y |
| Exposure | Appendix | | | | | | | | | | | | |
| | A for | | | | | | | | | | | | |
| | Formula | | | | | | | | | | | | |
| Test Strip | Water | ** | ** | *** | *** | * | * | * | * | ** | *** | *** | — |
| Indicator vs. | Hardness | | | | | | | | | | | | |
| Positive | (Ca + Mg) | | | | | | | | | | | | |
| Control | Copper | *** | *** | *** | *** | ** | ** | *** | *** | *** | *** | *** | — |
| (CHW) | Lead | ** | ** | * | * | * | * | * | * | * | * | ** | — |

TABLE 10B
Key for Table 10A
Key

| | |
|---|---|
| Significant Reduction vs. CHW Control | ** |
| Noticeable Reduction vs. CHW Control | ** |
| Slight Reduction vs. CHW Control | * |
| No Reduction vs. CHW Control | NR |
| CHW Control | — |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the disclosure. It is intended that the following claims define the scope of the present disclosure and that methods and structures within the scope of these claims and their equivalents be covered thereby.

What is claimed:

1. A hair composition, comprising:
(a) trisodium dicarhoxymethyl alaninate (MGDA), sodium phytate or phytic acid, sodium gluconate, tetrasodium glutamate diacetate (GLDA), or tetrasodium ethylenediaminetetraacetic acid (EDTA), or a combination of two or more thereof; and
(b) sodium carboxymethyl inulin, xanthan gum, hydroxymethylcellulose, hydroxyethylcellulose, cellulose, cellulose gum, microcrystalline cellulose, carboxymethylcellulose, pullula.n, sclerotium gum, tetragonoloba (guar) gum. acacia senegal gum, hydroxypropyl starch phosphate, lithium magnesium sodium silicate, or sodium magnesium fluorosilicate, or a combination of two or more thereof.

2. The hair composition of claim 1, wherein the hair composition has a pH from about6 to about 9.

3. The hair composition of claim 1, wherein the hair composition has a pH from about 7 to about 8.

4. The hair composition of claim 1, comprising trisodium MGDA and sodium phytate; trisodium MGDA and phytic acid; trisodium MGDA and sodium gluconate; trisodium MGDA and tetrasodium GLDA; trisodium MGDA and tetrasodium EDTA; sodium phytate and sodium gluconate; sodium phytate and tetrasodium GLDA; sodium phytate and tetrasodium EDTA; phytic acid and sodium gluconate; phytic acid and tetrasodium GLDA; phytic acid and tetrasodium EDTA; sodium gluconate and tetrasodium GLDA; sodium gluconate and tetrasodium EDTA; or tetrasodium EDTA and tetrasodium GLDA.

5. The hair composition of claim 1, comprising trisodium MGDA, sodium phytate, and sodium gluconate; sodium phytate, sodium gluconate, and tetrasodium GLDA; sodium gluconate, tetrasodium GLDA, and tetrasodium EDTA; sodium gluconate, tetrasodium GLDA, and tetrasodium EDTA; trisodium MGDA, tetrasodium GLDA, and tetrasodium EDTA; trisodium MGDA, sodium gluconate, and tetrasodium EDTA; or tri sodium MGDA, sodium gluconate, and tetrasodium GLDA.

6. The hair composition of claim 1, comprising trisodium MGDA, sodium phytate, phytic acid, and sodium gluconate; trisodium MGDA, sodium phytate, sodium gluconate, and tetrasodium GLDA; trisodium MGDA, sodium gluconate, tetrasodium GLDA, and tetrasodium EDTA; sodium phytate, sodium gluconate, tetrasodium GLDA, and tetrasodium EDTA; or tetrasodium EDTA, trisodiurn EDTA, disodium EDTA, and sodium gluconate.

7. The hair composition of claim 1, comprising pentasodium pentetate, trisodium ethylenediamine succinate, sodium thiosulfate, caprylhydroxamic acid, diiospropyl oxalate, disodium EDTA-copper, hydroxyethylethylenediaminetriacetic acid (HEDTA), oxalic acid, potassium citrate, sodium citrate, sodium oxalate, tris(2-hydroxyethyl)ammonium trihydrogen ethylenediaminetetraacetate (TEA-EDTA), trisodium HEDTA, or a combination of two or more thereof.

8. The hair composition of claim 1, comprising an antioxidant.

9. The hair composition of claim 8, wherein the antioxidant comprises a peptide antioxidant.

10. The hair composition of claim 9, wherein the peptide antioxidant comprises carnosine, valine-cysteine, di-proline, or di-phenylalanine, or a combination of two or more thereof.

11. The hair composition of claim 1, comprising a surfactant.

12. The hair composition of claim 11, wherein the surfactant comprises polysorbate 20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, or sodium surfactin, or a combination of two or more thereof.

13. The hair composition of claim 1, comprising lactic acid, citric acid, malic acid, gluconic acid, glucuronic acid. glycolic acid, tartaric acid, azelaic acid, acetic acid, sodium hydroxide, triethanolamine, or L-arginine, or a combination of two or more thereof.

14. The hair composition of claim 1, wherein the trisodium MGDA, sodium phytate, sodium gluconate, tetrasodium GLDA, or tetrasodium EDTA, or a combination of two or more thereof, is present at a total concentration of about 2% to about 15% (%w/w) of the hair composition.

15. The hair composition of claim 1, wherein the sodium carboxymethyl inulin, xanthan gum, hydroxyethylcellulose, hydroxymethylcellulose, cellulose, cellulose gum, microcrystalline cellulose, carboxymethylcellulose, pullulan, sclerotium gum, tetragonoloba (guar) gum, acacia senegal gum, hydroxypropyl starch phosphate, lithium magnesium sodium silicate, or sodium magnesium fluorosilicate, or a combination of two or more thereof, is present at a total concentration of about 0.1% to about 3% (%w/w) of the hair composition.

16. A method for removing a metal from hair, the method comprising:
contacting the hair with a hair composition comprising:
(a) trisodium dicarboxymethyl alaninate, sodium phytate, sodium gluconate, tetrasodium glutamate diacetate, or tetrasodium EDTA, or a combination of two or more thereof; and
(b) sodium carboxymethyl inulin, xanthan gum, hydroxymethylcellulose, hydroxyethylcellulose, cellulose, cellulose gum, microcrystalline cellulose, carboxymethylcellulose, pullulan, sclerotium gum, tetragonoloba (guar) gum, acacia senegal gum, hydroxypropyl starch phosphate, lithium magnesium sodium silicate, or sodium magnesium fluorosilicate, or a combination of two or more thereof;
binding the trisodium dicarboxymethyl alaninate, sodium phytate, sodium gluconate, tetrasodium glutamate diacetate, or tetrasodium EDTA, or a combination of two or more thereof, to the metal in the hair of the subject to form a complex; and
removing the complex, thereby removing the metal from the hair.

17. The method of claim 16, wherein the metal comprises calcium, magnesium, copper, iron, chromium, nickel, aluminum, lead, zinc, cadmium, mercury, or arsenic, or a combination of two or more thereof.

18. The method of claim 16, wherein the hair composition has a pH from about 6 to about 9.

19. The method of claim 16, wherein the hair composition has a pH from about 7 to about 8.

20. The method of claim 16, wherein the hair composition comprises tri sodium MGDA and sodium phytate; trisodium MGDA and phytic acid; trisodium MGDA and sodium gluconate; trisodium MGDA and tetrasodium GLDA; trisodium MGDA and tetrasodium EDTA; sodium phytate and sodium gluconate; sodium phytate and tetrasodium GLDA; sodium phytate and tetrasodium EDTA; phytic acid and sodium gluconate; phytic acid and tetrasodium GLDA; phytic acid and tetrasodium EDTA; sodium gluconate and tetrasodium GLDA, sodium gluconate and tetrasodium EDTA; or tetrasodium EDTA and tetrasodium GLDA.

21. The method of claim 16, wherein the hair composition comprises trisodium MGDA, sodium phytate, and sodium gluconate; sodium phytate, sodium gluconate, and tetrasodium GLDA; sodium gluconate, tetrasodium GLDA, and tetrasodium EDTA; sodium gluconate, tetrasodium GLDA, and tetrasodium EDTA; trisodium MGDA, tetrasodium GLDA, and tetrasodium EDTA; trisodiutn MGDA, sodium gluconate, and tetrasodium EDTA; or trisodium MGDA, sodium gluconate, and tetrasodium GLDA.

22. The method of claim 16, wherein the hair composition comprises tri sodium MGDA, sodium phytate, phytic acid, and sodium gluconate; trisodium MGDA, sodium phytate, sodium gluconate, and tetrasodium GLDA; trisodium MGDA, sodium gluconate, tetrasodium GLDA, and tetra.sodium EDTA; sodium phytate, sodium gluconate, tetrasodium GLDA, and tetrasodium EDTA; or tetrasodium EDTA, trisodium EDTA, disodium EDTA, and sodium gluconate.

23. The method of claim 16, wherein the hair composition comprises pentasodium pentetate, trisodium ethylenediamine succinate, sodium thiosulfate, caprylhydroxamic acid, diiospropyl oxalate, disodium EDTA-copper, hydroxyethylethylenediaminettiacetic acid (HEDTA), oxalic acid, potassium citrate, sodium citrate, sodium oxalate, tris(2-hydroxyethypammonium trihydrogen ethyl enediaminetetraacetate (TEA-EDTA), trisodium HEDTA, or a combination of two or more thereof.

24. The method of claim 16, wherein the hair composition comprises an antioxidant.

25. The method of claim 24, wherein the antioxidant comprises a peptide antioxidant.

26. The method of claim 25, wherein the peptide antioxidant comprises carnosine, valine-cysteine, di-proline, or di-phenylalanine, or a combination of two or more thereof.

27. The method of claim 16, comprising a surfactant.

28. The method of claim 16, wherein the hair composition comprises polysorbate 20, polyglyceryl-10 laurate, polyglyceryl-6 caprylate, polyglyceryl-3 cocoate, polyglyceryl-4 caprate, polyglyceryl-6 ricinoleate, pol.vglvceryl-6 caprylate, or sodium surfactin, or a combination of two or more thereof.

29. The method of claim 16, wherein the hair composition comprises lactic acid, citric acid, malic acid, gluconic acid, glucuronic acid, glycolic acid, tartaric acid, azelaic acid, acetic acid, sodium hydroxide, triethanolamine, or L-arginine, or a combination of two or more thereof.

30. The method of claim 16, wherein the trisodium dicarboxymethyl alaninate, sodium phytate, sodium gluconate, tetrasodium glutamate diacetate, or tetrasodium EDTA, or a combination of two or more thereof, is present at a total concentration of about 2% to about 15% (%w/w) of the hair composition.
